(19) United States
(12) Patent Application Publication
(10) Pub. No.: US 2016/0339241 A1
(43) Pub. Date: Nov. 24, 2016

(54) METHODS AND APPARATUS FOR ELECTRICAL STIMULATION

(71) Applicant: CEREPHEX CORPORATION, Los Altos, CA (US)

(72) Inventors: Jeffrey B. HARGROVE, Bancroft, MI (US); William G. MAVITY, Los Altos, CA (US); Lauren R. BRIGGS, Portland, MI (US); Roger Michael GENGREAU, Poway, CA (US)

(21) Appl. No.: 15/110,992

(22) PCT Filed: Jan. 21, 2015
(86) PCT No.: PCT/US15/12137

§ 371 Date: Jul. 12, 2016

Related U.S. Application Data
(60) Provisional application No. 61/929,917, filed on Jan. 21, 2014.

(51) Int. Cl. A61N 1/36; A61N 1/04; A61B 5/0476

(52) U.S. Cl. CPC A61N 1/36025; A61N 1/36021; A61B 5/0476; A61N 1/0456; A61N 2001/34

(57) ABSTRACT

A method of treating a neurological condition in a patient, the method including the step of applying an electrical stimulation from conductors to the patient's head at a stimulation application site. In some embodiments, the electrical stimulation includes a composite electrical signal further comprising at least one signal form configured to provide long-term treatment of the neurological condition and at least one signal form configured to provide analgesia for short-term pain relief. The invention also provides an electrical stimulation apparatus having an electrical signal generator adapted to provide an electrical signal form configured to provide long-term treatment of a neurological condition and to provide an electrical signal form configured to provide analgesia for short-term pain relief.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates generally to methods and apparatus for electrically stimulating a tissue. More specifically, the present invention relates to methods and apparatuses for using electrical stimulation to treat fibromyalgia and other neurological conditions involving central pain, central sensitivity and abnormal brain neural network connectivity.

### BACKGROUND

Electrical stimulation is known to alter the activity of the brain and modulate neurotransmitter activity, both of which are capable of providing therapeutic effects in both nociceptive pain conditions and central pain conditions. The use of electrical stimulation to treat pain and other neurological conditions is described in U.S. Pat. No. 7,715,910; U.S. Pat. No. 8,494,625; US Patent Publ. No. 2011/0307029; and WO 2013/169838. The disclosures of these publications are incorporated herein by reference.

Nociceptive pain is known to arise from stimulation of peripheral nerve endings. The peripheral nociceptive signal is transmitted through the spinal cord to the brain, where it is processed through numerous pain-processing networks. Descending pathways from the brain to the spinal cord subsequently modulate pain signals, thereby increasing or decreasing pain perception.

However, it is also known that enhanced activation of central pain-processing pathways and networks, through mechanisms such as neuroplastic changes in central neuronal activity and neural network connectivity, can lead to spontaneous pain in the absence of peripheral nociceptive input. When this occurs, pain is said to have “centralized”, which results in lower pain thresholds, secondary hyperalgesia in uninjured areas, and sustained pain potentiation. Brain-related centralized pain is thought to play a prominent role in chronic pain conditions.

Centralized pain is generally thought of as an outcome of central sensitivity (CS), which is also known as central sensitization, central augmentation, and central hypersensitivity among other terms. CS mechanisms in the brain have been implicated in the pathology of allodynia, which is the term used to describe a condition where pain is caused by a stimulus that does not normally provoke pain. CS mechanisms in the brain have also been implicated in hyperalgesia, which is the term used to describe a condition in which pain perceived from a stimulus is greater than what would normally be expected from that stimulus. Put simply, in central sensitivity the brain magnifies painful stimuli and eventually magnifies even associated non-painful stimuli. As pointed out in Latremoliere and Woolfe (1), because CS results from changes in the properties of neurons in the central nervous system, the pain is no longer coupled, as acute nociceptive pain is, to the presence, intensity, or duration of noxious peripheral stimuli arising from both neuropathic and inflammatory sources. Further, in chronic pain conditions the increased excitability caused by CS far outlasts the initiating noxious stimulus, that is, the nociceptive input that causes the pain to occur in the first place.

Before CS was discovered, typically only two models of pain were contemplated. The first is the aforementioned nociceptive pain model, by which specific pain pathways are activated by peripheral pain stimuli, and the amplitude and duration of the pain experienced is determined entirely by the intensity and timing of the peripheral pain inputs. The second model contemplates gate controls in the central nervous system that open and close, thus enabling or preventing pain. Medical science now recognizes CS as a third and unique model that contemplates neuroplastic changes in the functional properties and network connectivity of the central nervous system. For example, the level of resting brain activity within multiple networks (e.g., functional network connectivity and effective network connectivity) is now known to be associated with spontaneous pain in patients having centralized pain (2, 3). CS leads to reductions in pain threshold, increases in the magnitude and duration of responses to noxious input, and permits normally innocuous inputs to generate pain sensations. In addition, CS is also believed to be relevant in somatic symptoms associated with painful conditions, including but not limited to fatigue and sleep disorders.

The brain's role in CS is being increasingly revealed and understood in neuroscience, due in large part to the advent of functional brain imaging technologies. For example, Lee et al. (4) used functional magnetic resonance imaging (fMRI) to examine the extent to which brain activity contributes to the maintenance of CS in humans. When the intensity of pain during CS was matched to the intensity of pain during normal states, activity within the brainstem, including the mesencephalic pontine reticular formation and the anterior thalami, remained at an increased level during CS. Regarding brain areas related to the consequence of increased pain perception during CS, cortical activity, mainly in the primary somatosensory area, has been significantly correlated with the intensity of pain attributable to both the force of noxious stimulation used, and the state in which noxious stimulation was applied.

Borsook et al. (5) reviewed the literature on brain activity using neuroimaging technologies. Their review details evidence of alterations in multiple sub-cortical and cortical processing mechanisms. Those alterations include sensory, emotional/affective, cognitive, and modulatory systems that are present in chronic pain. The authors note these findings provide evidence that increases understanding of the importance of the role of numerous brain regions in the centralization of pain and the contributions of those regions to the altered brain states associated with chronic pain conditions. Similarly, Schweinhardt and Bushnell (6) reviewed neuroimaging evidence of the active and enhanced modulatory role that the brain plays in pain processing in chronic pain patients. Schweinhardt and Bushnell also cite findings that brain activations in chronic pain involve brain circuitry not normally activated by acute nociceptive pain.

Because of this emerging understanding, the role of CS is increasingly being shown to be pathological in seemingly unrelated chronic pain conditions and syndromes including fibromyalgia, complex regional pain syndrome, phantom pain, and migraine headaches. Yunus (7) identifies no less than 14 common syndromes that lack structural pathology yet have CS as a common mechanism. These conditions further include chronic fatigue syndrome, irritable bowel syndrome, tension-type headaches, temporomandibular disorder, myofascial pain syndrome, regional soft-tissue pain syndrome, restless leg syndrome, periodic limb movements in sleep, multiple chemical sensitivity, primary dysmenorrhea, female urethral syndrome, interstitial cystitis, and post-traumatic stress disorder. Yunus also notes that CS may play a significant role in the pain associated with depression and in Gulf War Syndrome.

Giesecke et al. (8) used fMRI to demonstrate augmented central pain processing in patients with idiopathic chronic low back pain and fibromyalgia. Indeed, when equal levels of mechanical pressure intended to elicit a painful response were applied to patients and to normal controls, patients with chronic low back pain and fibromyalgia experienced significantly more pain and showed more extensive, common patterns of neuronal activation in pain-related cortical areas of the brain than did the controls. Thus, CS may play an important role in persons with chronic low back pain that persists without identifiable physical pathology.

The role of CS in persistent inflammatory conditions is also gaining recognition. In Gwilym et al. (9), fMRI illustrated significantly greater brain activation in osteoarthritis (OA) patients in response to stimulation of their referred pain areas (i.e., areas where pain persists but do not exhibit OA or related inflammation) compared with healthy controls, and the magnitude of this activation positively correlated with the extent of neuropathic-like elements to the patient's pain. The role of CS in osteoarthritis has been the subject of several other investigations (10, 11, 12). As detailed in Imamura et al. (13), the refractory, disabling pain associated with knee OA is usually treated with total knee replacement. However, a comparison of OA patients with healthy normal controls showed patients with knee OA had significantly lower pressure pain thresholds (PPT) over widespread evaluated structures beyond the knee. The lower PPT values were correlated with higher pain intensity, higher disability scores, and with poorer quality of life. This suggests that pain in these patients might be more associated with CS than with peripheral inflammation and injury. As the authors point out, the implications of the role of CS, and its potential for modulation, may provide exciting and innovative cost effective therapeutic tools to control pain, reduce disability, and improve quality of life in knee OA patients.

Yet, the treatment of CS is a challenging task. As stated by Latremoliere and Woolfe (1), “The complexity is daunting because the essence of central sensitization is a constantly changing mosaic of alterations in membrane excitability, reductions in inhibitory transmission, and increases in synaptic efficacy, mediated by many converging and diverging molecular players on a background of phenotypic switches and structural alterations.” Some centrally-acting pharmaceutical agents such as gabapentin (14,15), ketamine (16), propofol (17) and anti-tumor necrosis factor alpha (TNF-alpha) therapy (18), just to name a few, have evidence of efficacy in treating CS. The patent literature has examples in the art of pharmaceutical use as a therapeutic agent for treating CS. For example, the use of dimiracetam for treatment of hyperalgesia and allodynia caused by central sensitization in chronic pain has been taught. Further, the use of compounds associated with (R)-2-acetamido-N-benzyl-3-methoxypropionamide has been taught to treat central neuropathic pain, including “neurological disorders characterized by persistence of pain and hypersensitivity in a body region.”

Another relevant consideration is that analyses of numerous brain imaging and functional measures, including electroencephalographic (EEG) measures (19), have been shown to produce measures related to brain networks and functional connectivity that correlate to findings produced by fMRI imaging (20). Thus, the presence of brain activity associated with CS, and hence centralized pain, can be determined using EEG measures and analysis.

In addition to abnormalities in brain neural network connectivity, it is also known that a number of neurotransmitters are involved in the processing of both central pain and nociceptive pain. In some cases, certain neurotransmitters augment the processing of pain and are therefore associated with increased pain. In other cases, certain neurotransmitters reduce the processing of pain, and are therefore associated with decreased pain.

The following citations are incorporated by reference in their entirety:
1. “Central sensitization: a generator of pain hypersensitivity by central neural plasticity”, Latremoliere A, Woolf C J. J Pain. 2009 September; 10(9):895-926.
2. “Intrinsic brain connectivity in fibromyalgia is associated with chronic pain intensity”, Napadow V, LaCount L, Park K, As-Sanie S, Clauw D J, Harris R E. Arthritis Rheum. 2010 August; 62(8):2545-55.
3. “Disrupted functional connectivity of the pain network in fibromyalgia”, Cifre I, Sitges C, Fraiman D, Muñoz M Á, Balenzuela P, González-Roldán A, Martinez-Jauand M, Birbaumer N, Chialvo D R, Montoya P. Psychosom Med. 2012 January; 74(1):55-62.
4. “Identifying brain activity specifically related to the maintenance and perceptual consequence of central sensitization in humans”, Lee M C, Zambreanu L, Menon D K, Tracey I. J Neurosci. 2008 Nov. 5; 28(45):1142-9.
5. “A key role of the basal ganglia in pain and analgesia—insights gained through human functional imaging”, Borsook D, Upadhyay J, Chudler E H, Becerra L. Mol Pain. 2010 May 13; 6:27.
6. “Pain imaging in health and disease—how far have we come?”, Schweinhardt P, Bushnell M C. J Clin Invest. 2010 Nov. 1; 15(11):3788-97.
7. “Fibromyalgia and overlapping disorders: the unifying concept of central sensitivity syndromes”, Yunus MB. Semin Arthritis Rheum. 2007 June; 36(6):339-56.
8. “Evidence of augmented central pain processing in idiopathic chronic low back pain”, Giesecke T, Gracely R H, Grant M A, Nachemson A, Petzke F, Williams D A, Clauw D J. Arthritis Rheum. 2004 February; 50(2):613-23.
9. “Psychophysical and functional imaging evidence supporting the presence of central sensitization in a cohort of osteoarthritis patients”, Gwilym S E, Keltner J R, Warnaby C E, Can A J, Chizh B, Chessell I, Tracey I. Arthritis Rheum. 2009 Sep. 15; 61(9):176-34.
10. “Lessons from fibromyalgia: abnormal pain sensitivity in knee osteoarthritis”, Bradley L A, Kersh B C, DeBerry J J, Deutsch G, Alarcón G A, McLain D A. Novartis Found Symp. 2004; 260:258-70.
11. “Sensitization in patients with painful knee osteoarthritis”, Arendt-Nielsen L, Nie H, Laursen M B, Laursen B S, Madeleine P, Simonsen O H, Graven-Nielsen T. Pain. 2010 June; 149(3):573-81.
12. “Pain mechanisms in osteoarthritis: understanding the role of central pain and current approaches to its treatment”, Mease P J, Hanna S, Frakes E P, Altman R D. J Rheumatol. 2011 August; 38(8):1546-51.
13. “Impact of nervous system hyperalgesia on pain, disability, and quality of life in patients with knee osteoarthritis: a controlled analysis”, Imamura M, Imamura S T, Kaziyama H H, Targino R A, Hsing W T, de Souza L P, Cutait M M, Fregni F, Camanho G L. Arthritis Rheum. 2008 Oct. 15; 59(10):1424-31.
14. “Pharmacological modulation of pain-related brain activity during normal and central sensitization states in humans”, Iannetti G D, Zambreanu L, Wise R G, Buchanan T J, Huggins J P, Smart T S, Vennart W, Tracey I. Proc Natl Acad Sci USA. 2005 Dec. 13; 102(50):18195-200.
15. “Chronic oral gabapentin reduces elements of central sensitization in human experimental hyperalgesia”, Gottrup H, Juhl G, Kristensen A D, Lai R, Chizh B A, Brown J, Bach F W, Jensen T S. Anesthesiology. 2004 December; 101(6):350-8.
16. “Pharmacodynamic profiles of ketamine (R)- and (S)- with 5-day inpatient infusion for the treatment of complex regional pain syndrome”, Goldberg M E, Torjman M C, Schwartzman R J, Mager D E, Wainer I W. Pain Physician. 2010 July; 13(4):379-87.
17. “Analgesic and antihyperalgesic properties of propofol in a human pain model”, Bandschapp O, Filitz J, Ihmsen H, Berset A, Urwyler A, Koppert W, Ruppen W. Anesthesiology. 2010 August; 8(2):421-8.
18. “TNF-alpha and neuropathic pain—a review”, Leung L, Cahill C M. J Neuroinflammation. 2010 Apr. 16; 7:27.
19. “Functional connectivity: the principal-component analysis of large (PET) data sets”, Friston K J, Frith C D, Liddle P F, Frackowiak R S. J Cereb Blood Flow Metab 1993; 13:5-14.
20. “Electrophysiological signatures of resting state networks in the human brain”, Mantini D, Perrucci M G, Del Gratta C, Romani G L, Corbetta M. Proc Natl Acad Sci USA. 2007 Aug. 7; 104(32):2670-5.

### SUMMARY OF THE DISCLOSURE

The use of amplitude-modulated, pulse-width modulation signals to treat neurological dysfunction via stimulation of the brain is described, e.g., in U.S. Pat. No. 7,715,910. Clinical tests have shown that specific electrical stimulation parameters—including waveform parameters, treatment protocols and stimulation sites—can provide significant therapy benefits. These discoveries have also led to the development of stimulation systems particularly suited to providing efficacious electrical stimulation therapy.

In the following description of the disclosed methods and apparatus, the term “centralized pain” is intended to mean any form of pain, whether chronic or acute, that is enhanced in its characteristics; such as magnitude, duration and scope; due to abnormal brain activity associated with pain processing, or abnormalities involving neurotransmitters associated with pain. Such brain activity may include, but is not limited to, central sensitivity and network connectivity. Such neurotransmitters may include, but are not limited to, serotonin, norepinephrine, glutamate, N-Methyl-D-aspartic acid, Substance P, gama-aminobutyric acid, various nucleotides and dopamine.

The term “central sensitivity” is intended to mean any central nervous system condition pathologically related to hyperalgesia, allodynia, reductions in pain threshold, increases in the magnitude and duration of responses to noxious input, results in normally innocuous inputs to generate pain sensations, or results in non-painful symptoms associated with increases in central nervous system responsiveness. Central sensitivity is also known by alternate terms that include but are not limited to “central sensitization”, “central pain”, “central augmentation,” and “central hypersensitivity”.

The terms “network connections” and “network connectivity” are intended to mean various forms of relationships between neural networks in brain regions involved in processing of information such as pain. For example, “functional connectivity” refers to a statistical correlation between the activities of different brain regions. “Effective connectivity” denotes not simply a statistical but a causal influence between two brain regions.

The terms “treat” or “treating” are intended to mean the act of reducing, making less severe, mitigating, alleviating, or eliminating a condition and/or its symptoms for any period of time.

Except where the context requires otherwise, the term “comprise” and variations of the term, such as “comprising”, “comprises” and “comprised” are not intended to be exclusive. Where, for example, a form of the word “comprise” is used to refer to one or more additives, components, integers or steps; its use is not intended to exclude other additives, components, integers or steps.

The term “stimulating” is intended to mean the transmitting of any energy signal generated by a stimulation device, such as an electrical stimulator, to the brain of a subject for the purpose of influencing any function or physiological state of the subject's brain that is at least one part of a pathway of pain; or for the purpose of influencing at least one neurotransmitter associated with pain.

The term “stimulation signal” is intended to mean any energy signal used in the process of stimulating a tissue such as a brain. Other terms used to refer to such a signal may include but are not limited to “cranial neurostimulation”, “cortical stimulation”, “neuromodulation” and “neurostimulation”.

The term “nociceptive pain” is intended to mean any pain arising from stimulation of peripheral nerve endings.

The term “conductor” is intended to include any electrical conduction pathway apparatus, such as electrical leads comprising wires, between an apparatus for providing electrical stimulation and a tissue to be stimulated. The term “conductor” also includes an electrode or any other terminating feature at the end of a conduction pathway apparatus configured to interface to the tissue to be stimulated.

The term “waveshape” is intended to mean an electrical signal waveform that may be approximated by an amplitude envelope resulting from the peaks of an amplitude modulated pulse train.

The term “analgesia” is intended to mean any reduction or elimination in the amount, intensity, duration, location, form of, or perception of pain.

The term “rest” is intended to mean periods of time which are part of therapy and in which a patient is in a state of preparedness to receive an electrical stimulation signal, but no signal is actually being delivered to the patient.

The electrical stimulation signal forms taught herein have been studied in clinical trials involving patients with fibromyalgia. Clinical data arising from these studies have demonstrated symptom improvement and evidence that central nervous system pain processing mechanisms are modified. Thus, the approach may hold promise for the treatment of numerous chronic pain states where abnormal central pain processing mechanisms are involved.

A method is taught for treating a neurological condition in a patient. Such neurological conditions may include, but are not limited to, fibromyalgia, hyperalgesia, central pain, central sensitivity, chronic pain, abnormal brain network connectivity, neuropathic pain, central pain arising from chronic osteoarthritis, central pain arising from chronic back pain, chronic headache, migraine headache or depression.

The method may include the step of applying an electrical stimulation signal from conductors to the patient's head at a stimulation application site. The electrical stimulation signal may include a tissue transmission component and a therapeutic component. The method may also include the step of sequencing the electrical stimulation signal in bursts. One such sequence may include at least two burst periods with at least one rest period in between each pair of bursts in which no stimulation is provided. In the method, the electrical stimulation signal may be subthreshold for detection by the patient. Further, the tissue transmission component may include a pulse train that has pulse frequencies sufficient to reduce tissue impedance between the conductors and the patient's brain. In one embodiment, the pulse train amplitude may have a minimum value of 0 volts and a maximum value of 1 volt. In another embodiment, the pulse train amplitude may have a maximum value of 0.2 volts. In another embodiment, the pulse train frequency is between 10,000 Hz and 20,000 Hz. In another embodiment, the pulse train frequency is approximately 15,000 Hz. The pulse train may also be monopolar.

Further to the method, the pulse train may be pulse width modulated to create a variable duty cycle of on time and off time. In one embodiment, the pulse train duty cycle may be between 20% and 60%. In another embodiment, the pulse train duty cycle may be approximately 37.5%. Further still, the pulse train may be amplitude modulated to create a waveshape which may include an amplitude envelope. Such waveshapes may include those that are formed to create a therapeutic component. In one embodiment, the amplitude envelope may form a rectangular wave. In another embodiment, the amplitude envelope may form a sinusoidal wave. In another embodiment, the amplitude envelope may form a composite of multiple sinusoidal waves. In one embodiment, the waveshape may have a frequency between 1 Hz and 30 Hz. In another embodiment, the waveshape may have a frequency between 7 Hz and 12 Hz.

In another embodiment, the waveshape is a rectangular wave with frequencies ranging from 7 Hz to 12 Hz as a function of time. In another embodiment, multiple sinusoidal waves may have individual frequencies between 1 Hz and 30 Hz. Further to the method, the frequency of the waveshape may change as a function of stimulation delivery time.

In one embodiment of the method, a pulse train may have amplitude with minimum value of 0 volts and maximum value of 0.2 volts, a frequency of approximately 15,000 Hz and a duty cycle of approximately 37.5%.

Further to the method, stimulation may be applied in bursts with periods of time ranging from 1 second to 5 minutes. In one embodiment, each burst period may range in time from 30 seconds to 2 minutes. Further still, each rest period may range in time from 1 second to 5 minutes. In one embodiment, each rest period may be 60 seconds.

Further still to the method, the patient's electroencephalogram (EEG) signal may be measured, including times when EEG may be measured during a rest period. In one embodiment, the EEG may be measured at the stimulation application site. In another embodiment, the patient's EEG signal may be measured during a period of rest prior to a first burst of stimulation signal. In an embodiment, such periods of rest prior to a first burst may range in time from 1 second to 5 minutes. In another embodiment, such period of rest prior to a first burst may be 3 minutes.

Further still to the method, the patient's EEG signal may be measured during a period of rest after a final burst of stimulation signal. In an embodiment, such periods of rest after a final burst may range in time from 1 second to 5 minutes. In another embodiment, such period of rest after a final burst may be 3 minutes.

Further still to the method, the sequencing step may include a burst and rest time sequence consisting of a first three minute period of rest; then applying a first burst of the electrical stimulation signal for 30 seconds; then after applying the first burst, ceasing application of the electrical stimulation signal for a second period of rest lasting 60 seconds; then after the second period of rest, applying a second burst of the electrical stimulation signal for 60 seconds; then after applying the second burst, ceasing application of the electrical stimulation signal for a third period of rest lasting 60 seconds; then after the third period of rest, applying a third burst of the electrical stimulation signal for 90 seconds; and then after applying the third burst, ceasing application of the electrical stimulation signal for a fourth period of rest lasting three minutes. In the embodiments, the stimulation burst may include a pulse train of pulses having amplitude with a minimum value of 0 volts, a maximum value of 0.2 volts, a frequency of approximately 15,000 Hz and a duty cycle of approximately 37.5%. Further, the stimulation signal burst maybe amplitude modulated to form rectangular waveshapes, with the waveshapes having frequencies sweeping from 7 Hz to 12 Hz over approximately equal periods of time during the burst.

Further still to the method, the applying step may include the step of placing conductors to create a current pathway through at least one portion of the patient's brain. Such portions of a brain may be selected from a group consisting of, but not limited to, the parietal lobes, somatosensory cortex, thalamus, prefrontal cortex, primary motor cortex, secondary motor cortex, insula or default mode network. In one embodiment, the conductors may be placed proximate to the portion of the brain to be stimulated. In another embodiment, a first conductor may be placed proximate to the parietal lobes along the median plane, and a second conductor may be placed proximate to the right ear. In another embodiment, a first conductor may be placed proximate to International 10-20 site Pz, and a second conductor may be placed proximate to the right ear lobe. In another embodiment, the conductors may be noninvasive.

The method may be further realized by performing the applying and sequencing steps repeatedly over a period of time. In one embodiment, the applying and sequencing steps may be performed at least once a day, with at least one day transpiring between applications. In another embodiment, the applying and sequencing steps may be performed twice in a calendar week, with two days transpiring between applications. In another embodiment, the applying and sequencing steps may be performed repeatedly over a period ranging from 8 to 24 consecutive weeks. In another embodiment, the applying and sequencing steps may be performed repeatedly over a period of 12 consecutive weeks. In another embodiment, the applying and sequencing steps may be performed 24 times over the period of time. In another embodiment, the applying and sequencing steps may be performed additional times to further treat the neurological condition and achieve more satisfactory alleviation of symptoms.

A method is taught for treating a neurological condition in a patient. Such neurological conditions may include, but are not limited to, fibromyalgia, hyperalgesia, central pain, central sensitivity, chronic pain, abnormal brain network connectivity, neuropathic pain, central pain arising from chronic osteoarthritis, central pain arising from chronic back pain, chronic headache, migraine headache or depression. The method may include placing a first conductor on the patient's head proximate to International 10-20 site Pz and placing a second conductor proximate to the patient's right ear lobe; then applying an electrical stimulation signal between the first and second conductors, where the electrical stimulation signal may include a tissue transmission component which may further include a monopolar pulse train of frequency sufficient to reduce tissue impedance between the conductors and the patient's brain, and where the pulse train amplitude may have a minimum value of 0 volts, a maximum value of 0.2 volts, a frequency of approximately 15,000 Hz and a duty cycle of approximately 37.5%, and where the pulse train may be amplitude modulated to form a rectangular waveshape with frequencies ranging from 7 Hz to 12 Hz as a function of time; then sequencing the application of the electrical stimulation signal in a burst and rest time sequence consisting of a first three minute period of rest; and then after the first period of rest, applying a first burst of the electrical stimulation signal for 30 seconds, with frequencies sweeping from 7 Hz to 12 Hz over approximately equal periods of time during the burst; and then after applying the first burst, ceasing application of the electrical stimulation signal for a second period of rest lasting 60 seconds; and then after the second period of rest, applying a second burst of the electrical stimulation signal for 60 seconds, with frequencies sweeping from 7 Hz to 12 Hz over approximately equal periods of time during the burst; and then after applying the second burst, ceasing application of the electrical stimulation signal for a third period of rest lasting 60 seconds; and then after the third period of rest, applying a third burst of the electrical stimulation signal for 90 seconds, with frequencies sweeping from 7 Hz to 12 Hz over approximately equal periods of time during the burst; and then after applying the third burst, ceasing application of the electrical stimulation signal for a fourth period of rest lasting three minutes; and then performing the placing, applying and sequencing steps twice in a calendar week, with at least one day transpiring between treatment applications, over a period of 12 consecutive weeks.

A method is taught for treating a neurological condition in a patient. Such neurological conditions may include, but are not limited to, fibromyalgia, central sensitivity, central pain, abnormal neural network connectivity, complex regional pain syndrome, phantom pain, irritable bowel syndrome, temporomandibular disorder, myofascial pain syndrome, regional soft-tissue pain syndrome, neuropathic pain, osteoarthritis, back pain, post-operative pain, depression, tension-type headaches or migraine headaches.

The method may include the step of applying an electrical stimulation from conductors to the patient's head at a stimulation application site, and in which the electrical stimulation may include a composite electrical signal which may further include at least one signal form configured to provide long-term treatment of the neurological condition and at least one signal form configured to provide analgesia for short-term pain relief.

In one embodiment, the at least one signal form configured to provide long-term treatment of the neurological condition and the at least one signal form configured to provide analgesia for short-term pain relief may be applied simultaneously during a treatment application. In another embodiment, the at least one signal form configured to provide long-term treatment of the neurological condition and the at least one signal form configured to provide analgesia for short-term pain relief may be applied at alternating times during a treatment application.

In one embodiment, the at least one signal form configured to provide long-term treatment of the neurological condition may include an electrical signal configured to treat an abnormal brain condition associated with the neurological condition. In another embodiment, the at least one signal form configured to provide analgesia for short-term pain relief may include an electrical signal configured to stimulate modulation of one or more neurotransmitters associated with analgesia. Further to the embodiment, the modulation may be selected from a group consisting of, but not limited to, the release, expression, uptake, increased production, reduced production, inhibition or elimination of neurotransmitters. Further to the embodiment, the neurotransmitters may be selected from a group consisting of, but not limited to, serotonin, norepinephrine, glutamate, N-Methyl-D-aspartic acid, Substance P, gama-aminobutyric acid, various nucleotides or dopamine.

In an embodiment of the method, the at least one signal form configured to provide long-term treatment of the neurological condition may include a tissue transmission component and a therapeutic component. Further to the embodiment, the tissue transmission component may include a pulse train of frequency sufficient to reduce tissue impedance between the conductors and the patient's brain. Further still, the pulse train amplitude may have a minimum value of 0 volts and a maximum value of 1 volt. In another embodiment, the pulse train amplitude may have a maximum value of 0.2 volts. In one embodiment, the pulse train frequency may be between 10,000 Hz and 20,000 Hz. In another embodiment, the pulse train frequency may be approximately 15,000 Hz. In the embodiments, the pulse train may be monopolar. Further to the embodiments, the electrical stimulation may be subthreshold for detection by the patient.

Further to the method, the pulse train may be pulse width modulated to create a variable duty cycle of on time and off time. In one embodiment, the pulse train duty cycle may be between 20% and 60%. In another embodiment, the pulse train duty cycle may be approximately 37.5%. Further still to the method, the pulse train may be amplitude modulated to create a waveshape which may include an amplitude envelope. In one embodiment, the waveshape may form a therapeutic component. In another embodiment, the amplitude envelope may form a rectangular wave. In another embodiment, the amplitude envelope may form a sinusoidal wave. In another embodiment, the amplitude envelope may form a composite of multiple sinusoidal waves. In one embodiment, the waveshape may have a frequency between 1 Hz and 30 Hz. In another embodiment, the waveshape may have a frequency between 7 Hz and 12 Hz. In another embodiment, the waveshape is a rectangular wave with frequencies ranging from 7 Hz to 12 Hz as a function of time. In another embodiment, multiple sinusoidal waves may have individual frequencies between 1 Hz and 30 Hz. Further to the method, the frequency of the waveshape may change as a function of stimulation delivery time.

In one embodiment of the method, a pulse train may have amplitude with minimum value of 0 volts and maximum value of 0.2 volts, a frequency of approximately 15,000 Hz and a duty cycle of approximately 37.5%.

Further to the method, stimulation may be applied in bursts with periods of time ranging from 1 second to 5 minutes. In one embodiment, each burst period may range in time from 30 seconds to 2 minutes. Further still, each rest period may range in time from 1 second to 5 minutes. In one embodiment, each rest period may be 60 seconds.

Further still, the electrical stimulation may include a sequencing step in which an electrical signal is applied in bursts for at least two burst periods with at least one rest period which may include no stimulation in between each pair of bursts. In one embodiment, each burst period may range in time from 1 second to 5 minutes. In another embodiment, each burst period may range in time from 30 seconds to 2 minutes. In one embodiment, each rest period may range in time from 1 second to 5 minutes. In another embodiment, each rest period may be 60 seconds.

Further still to the method, the patient's electroencephalogram (EEG) signal may be measured, including times when EEG may be measured during a rest period. In one embodiment, the EEG may be measured at the stimulation application site. In another embodiment, the patient's EEG signal may be measured during a period of rest prior to a first burst of stimulation signal. In an embodiment, such periods of rest prior to a first burst may range in time from 1 second to 5 minutes. In another embodiment, such period of rest prior to a first burst may be 3 minutes.

Further still to the method, the patient's EEG signal may be measured during a period of rest after a final burst of stimulation signal. In an embodiment, such periods of rest after a final burst may range in time from 1 second to 5 minutes. In another embodiment, such period of rest after a final burst may be 3 minutes.

Further still to the method, the sequencing step may include a burst and rest time sequence consisting of a first three minute period of rest; then applying a first burst of the electrical stimulation signal for 30 seconds; then after applying the first burst, ceasing application of the electrical stimulation signal for a second period of rest lasting 60 seconds; then after the second period of rest, applying a second burst of the electrical stimulation signal for 60 seconds; then after applying the second burst, ceasing application of the electrical stimulation signal for a third period of rest lasting 60 seconds; then after the third period of rest, applying a third burst of the electrical stimulation signal for 90 seconds; and then after applying the third burst, ceasing application of the electrical stimulation signal for a fourth period of rest lasting three minutes. In the embodiments, the stimulation burst may include a pulse train of pulses having amplitude with a minimum value of 0 volts, a maximum value of 0.2 volts, a frequency of approximately 15,000 Hz and a duty cycle of approximately 37.5%. Further, the stimulation signal burst maybe amplitude modulated to form rectangular waveshapes, with the waveshapes having frequencies sweeping from 7 Hz to 12 Hz over approximately equal periods of time during the burst.

Further to the method, the at least one signal form configured to provide analgesia for short-term pain relief may include a periodic signal. In one embodiment, the periodic signal may be a pulse train. In another embodiment, the periodic signal may be a sinusoidal waveform. In another embodiment, the periodic signal may have frequencies between 1 Hz and 300 Hz. In another embodiment, the periodic signal may have frequencies between 50 Hz and 150 Hz. In another embodiment, the periodic signal may have a frequency of approximately 60 Hz. In another embodiment, the periodic signal may have a frequency of approximately 120 Hz. In one embodiment, the periodic signal may have amplitudes between −10 volts and +10 volts. In another embodiment, the periodic signal may have a minimum amplitude of −1 volts and maximum amplitude of +1 volt.

Further to the method, the at least one signal form configured to provide analgesia for short-term pain relief may be applied during periods of rest time before or after bursts of at least one signal form configured to provide long-term treatment of a neurological condition. Further still, the at least one signal form configured to provide analgesia for short-term pain relief may include a direct current (DC) signal.

Further still to the method, the applying step may include the step of placing conductors to create a current pathway through at least one portion of the patient's brain. Such portions of a brain may be selected from a group consisting of, but not limited to, the parietal lobes, somatosensory cortex, thalamus, prefrontal cortex, primary motor cortex, secondary motor cortex, insula or default mode network. In one embodiment, the conductors may be placed proximate to the portion of the brain to be stimulated. In another embodiment, a first conductor may be placed proximate to the parietal lobes along the median plane, and a second conductor may be placed proximate to the right ear. In another embodiment, a first conductor may be placed proximate to International 10-20 site Pz, and a second conductor may be placed proximate to the right ear lobe. In another embodiment, the conductors may be noninvasive.

The method may be further realized by performing the applying and sequencing steps repeatedly over a period of time. In one embodiment, the applying and sequencing steps may be performed at least once a day, with at least one day transpiring between applications. In another embodiment, the applying and sequencing steps may be performed twice in a calendar week, with two days transpiring between applications. In another embodiment, the applying and sequencing steps may be performed repeatedly over a period ranging from 8 to 24 consecutive weeks. In another embodiment, the applying and sequencing steps may be performed repeatedly over a period of 12 consecutive weeks. In another embodiment, the applying and sequencing steps may be performed 24 times over the period of time. In another embodiment, the applying and sequencing steps may be performed additional times to further treat the neurological condition and achieve more satisfactory alleviation of symptoms.

Further to the method, the applying step may include applying electrical stimulation through conductors that create at least a first current pathway through at least a first portion of the patient's brain for providing long-term treatment of the neurological condition, and at least a second current pathway through at least a second portion of the patient's brain for providing analgesia for short-term pain relief. In one embodiment, the conductors may include at least two electrical leads placed proximate to the portion of the patient's brain to be stimulated. In another embodiment, the conductors may be noninvasive.

A method is taught for treating a neurological condition in a patient. Such neurological conditions may include, but are not limited to, fibromyalgia, hyperalgesia, central pain, central sensitivity, chronic pain, abnormal brain network connectivity, neuropathic pain, central pain arising from chronic osteoarthritis, central pain arising from chronic back pain, chronic headache, migraine headache or depression. The method may include placing a first electrical lead on the patient's head proximate to International 10-20 site Pz and placing a second electrical lead proximate to the patient's right ear lobe; and then applying a composite electrical stimulation signal which may include a signal configured to provide long-term treatment of the neurological condition and a signal configured to provide analgesia for short-term pain relief, where the signal configured to provide analgesia for short-term pain relief may include a pulse train of both 60 Hz positive pulses and 60 Hz negative pulses, where the positive pulses may have a minimum amplitude of 0 volts and a maximum amplitude of 0.5 volts, where the negative pulses may have a maximum amplitude of 0 volts and a minimum amplitude of −0.5 volts, where the positive and negative pulses may alternate in time and may be equally spaced in time, where the signal to provide long-term treatment of the neurological condition may include a monopolar pulse train of frequency sufficient to reduce tissue impedance, where the pulse train amplitude may have a minimum value of 0 volts, a maximum value of 0.2 volts, a frequency of approximately 15,000 Hz and a duty cycle of approximately 37.5%, and where the pulse train may be amplitude modulated to form a rectangular waveshape with frequencies which ranging from 7 Hz to 12 Hz as a function of time; and then sequencing application of the electrical stimulation signal in a burst and rest time sequence that may consist of a first burst which may include the signal configured to provide analgesia for short-term pain relief, lasting three minutes, then a second burst which may include the signal configured to provide long-term treatment of the neurological condition, lasting 30 seconds, with frequencies sweeping from 7 Hz to 12 Hz over approximately equal periods of time during the burst, and then a third burst which may include the signal configured to provide analgesia for short-term pain relief, lasting 60 seconds, and then a fourth burst which may include the signal configured to provide long-term treatment of the neurological condition, lasting 60 seconds, with frequencies sweeping from 7 Hz to 12 Hz over approximately equal periods of time during the burst, and then a fifth burst which may include the signal configured to provide analgesia for short-term pain relief, lasting 60 seconds, and then a sixth burst which may include the signal configured to provide long-term treatment of the neurological condition, lasting 90 seconds, with frequencies sweeping from 7 Hz to 12 Hz over approximately equal periods of time during the burst; and then a seventh burst which may include the signal configured to provide analgesia for short-term pain relief, lasting three minutes; and then conducting the electrical stimulation along a current pathway which may be created between the first electrical lead placed proximate to International 10-20 site Pz, and the second electrical lead placed proximate to the right ear lobe; and then performing the placing, applying and sequencing steps twice in a calendar week, with at least one day transpiring between treatment applications, over a period of 12 consecutive weeks.

An electrical stimulation apparatus is taught which may include an electrical signal generator adapted to provide an electrical signal form configured to provide long-term treatment of a neurological condition and to provide an electrical signal form configured to provide analgesia for short-term pain relief. In one embodiment, the electrical signal generator may further include at least one microcontroller configured to generate composite signal waveforms and coupled to at least one signal conditioning circuit configured to transform the composite signal waveforms into stimulation signals. Further to the embodiment, the at least one signal conditioning circuit may include an amplifier circuit.

In another embodiment, the electrical stimulation apparatus may further include any one or more circuit elements selected from the group of circuit elements consisting of, but not limited to, an EEG amplifier configured to measure EEG signals, a filter circuit configured to reduce electrical noise in EEG signals, an isolation amplifier configured to protect human subjects, an analog-to-digital interface configured to convert analog EEG signals to digital signals, and an isolated power supply configured to provide circuit power and human subject protection.

Further still, an embodiment of the apparatus may further include at least two electrical leads for providing a composite stimulation signal current pathway between the electrical signal generator and a tissue to be stimulated. In another embodiment, the apparatus may further include a user interface, which may further include a software graphic user interface which provides user guidance for providing a composite stimulation signal.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an apparatus for providing electrical stimulation according to an embodiment of the invention.

FIG. 2 shows an example composite stimulation signal featuring one signal form configured for providing long-term treatment of a neurological condition and a second signal form configured for providing analgesia for short-term pain relief.

FIG. 3 shows a block diagram of a composite electrical signal generator apparatus according to an embodiment of the invention.

FIG. 4 shows a block diagram of a composite electrical signal generator apparatus to manage timing of the therapy application according to an embodiment of the invention.

### DETAILED DESCRIPTION

While the use of electrical stimulation for therapeutic purposes has been taught through numerous examples in the art, the methods and apparatuses described herein provide several advantages and improvements over previously disclosed matter.

Disclosed herein is an electrical stimulation system involving forms of electrical stimulation signals and treatment delivery strategies that have been shown in clinical research trials to provide therapeutic advantages and lasting results in persons suffering from fibromyalgia and similar pain conditions involving central pain.

A key element of the electrical stimulation methods taught herein is the utilization of stimulation signal forms that include (1) a transmission component configured to enhance the ability of the signal to transmit through tissues to be stimulated, and to do so in a way that reduces attenuation due to tissue impedance; and (2) a therapeutic component configured to treat mechanisms involved in central pain, such as central sensitivity and abnormal neural network connectivity in the brain. In this invention, the therapeutic signal component may be configured to treat nociceptive pain or provide analgesia. Alternately, the therapeutic signal component may be configured to reduce aberrant brain function mechanisms such as those that process pain signals in ways that cause abnormal perception of pain. In other words, the methods taught herein target abnormal pain processing mechanisms, but may also treat nociceptive generators of pain or block pain signals ascending from peripheral nerves.

Such signal components configured to enhance signal transmission include the use of a pulse train of frequency high enough to reduce the electrical admittivity of intervening tissues between a conductor and the brain. In one embodiment, the pulse train is monopolar so as to deliver a positive net charge to the patient. It may also be pulse width modulated in a way that advantageously creates a variable duty cycle. This is done to provide control of the time averaged power delivered by the pulse train signal.

The method is further realized when the pulse train is amplitude modulated to create a waveshape comprising an amplitude envelope approximately following the peak values of the amplitude modulated pulses. The waveshape is created in a way that the amplitude envelope forms another periodic signal having at least one frequency lower than the frequency of the pulse train. Such periodic signals may be realized through any waveform, such as a rectangular wave, a sinusoidal wave, or a composite of multiple sinusoidal waves. The frequencies of the waveshape may be fixed, or they may change over time as the stimulation signal is being delivered. Advantages of changing frequencies over time may include the ability to provide (1) a broader range of frequency-dependent stimulation benefits in a single therapy application, and (2) a sequential adjustment of stimulation frequency that may prevent overstimulation of the brain at any particular frequency.

The lower frequency waveshape provides the stimulation signal's therapeutic component configured to treat mechanisms involved in central pain, such as central sensitivity and abnormal neural network connectivity in the brain. In combination, the signal component configured to enhance signal transmission and the therapeutic lower frequency waveshape provides a stimulation signal that can be effectively transmitted through tissues with lower driving voltages. The advantages of this include greater safety in the stimulation signal, and the ability to provide therapeutic electrical stimulation with signals that are subthreshold for detection by a patient being stimulated. In treating patients who are suffering from enhanced pain processing, and are therefore experiencing allodynia and/or hyperalgesia, the ability to stimulate without the signal itself causing pain or any negative sensation is a significant advantage.

The signal method may further include a signal component that is configured to provide analgesia for short-term pain relief. While a signal featuring components to reduce aberrant brain function mechanisms that process pain signals in ways that cause an abnormal perception of pain has long-term therapeutic utility, actual pain relief experienced by the patient may not be immediate. Thus, in some embodiments the method uses additional signal components that may, among other things, modulate neurotransmitters in the body to cause analgesia and short-term pain relief.

Such signals configured to provide analgesia may include a DC signal or a periodic signal such as pulse trains or sinusoidal waveforms. Such periodic signals may have frequencies between 1 Hz and 300 Hz, which is a range of frequencies sometimes known as the “physiologic stimulation range”. Other frequencies may be selected to stimulate certain neurotransmitters. For example, it is known that frequencies in the range of 50-150 Hz may stimulate the release of the pain mediating neurotransmitters dopamine, serotonin and norepinephrine. Such periodic signals may also have both positive and negative amplitudes ranges, such as for example between −1 volt and +1 volt.

The method is further realized when the electrical stimulation signals are delivered in a therapeutic setting, using repeat applications and over a period of time. The advantages of repeat applications over a period of time include the provision of therapy that modulates the abnormal pain processing mechanisms associated with fibromyalgia and other neurological conditions involving central pain, reinforces normal pain processing mechanisms, and therefore creates a lasting therapeutic benefit.

Further to the method, the repeat applications of therapy involve signal deliveries in bursts typically lasting anywhere from a few seconds to several minutes. Before, after and in between these bursts, the method involves periods of therapeutic rest in which the patient remains in a state for receiving stimulation, but no stimulation signal is actually delivered. Such state for receiving stimulation may include the patient being physically connected to conductors of an electrical stimulation apparatus, sitting still in a clinical environment during which time their eyes are closed and they are purposefully told to relax.

During such rest periods, the patient merely spends time in a state of relaxation and disengagement from general attentiveness to their surroundings. Providing such rest periods may be therapeutically advantageous in that the mechanisms associated with abnormal pain processing, for example hyperconnectivity in brain networks, may diminish or mitigate when a patient is in such state. When the stimulation bursts are subsequently applied, the stimulation may reinforce states such as a reduced connective state, and therefore reinforce the therapeutic benefit.

Further still to the method, the therapy applications may be provided over a period of several days per week and several weeks per a course of therapy. The advantage of such approach is again conceived to provide long-term therapeutic benefit. The method also anticipates a therapeutic advantage of providing at least one day in between therapy applications, during which time the patient will receive no stimulation therapy.

Further still to the method, the patient's electroencephalogram (EEG) signal may be measured at times consistent with the therapy application. In one embodiment, EEG may be measured during periods of rest. In another embodiment, EEG may be measured at the electrical stimulation signal application site. In yet another embodiment, EEG may be measured at a number of additional sites on the scalp. The uses for EEG measurement at the signal application site include means of assuring the integrity of electrical contact between conductors and the patient, and thus assuring quality stimulation signal delivery. The uses for EEG measurement at additional sites on the scalp include providing a means for assessing brain neural network connectivity before, during and after stimulation signal application, the benefits of which may include providing indication of the levels and characteristics of at least one parameter of abnormal pain processing mechanisms in the brain.

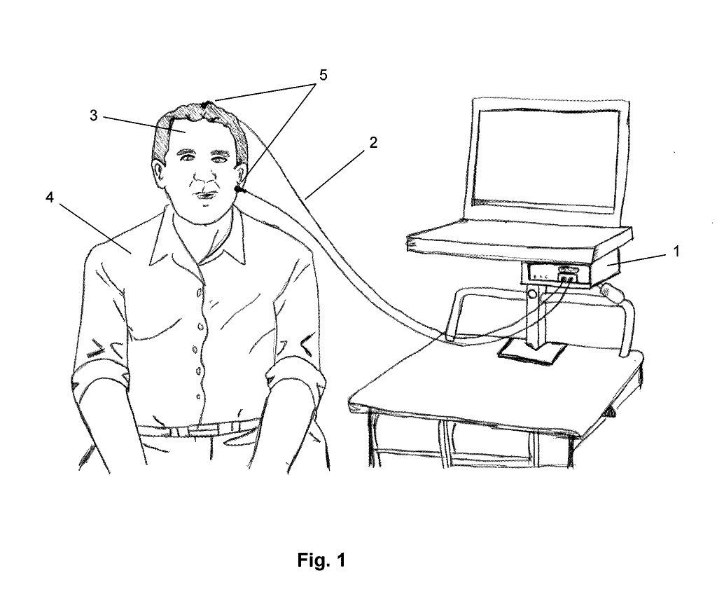

With reference to FIG. 1, the method may be practiced by using an apparatus 1 configured to generate electrical stimulation signals, and in particular the kinds of composite electrical stimulation signals disclosed herein. In one embodiment, such apparatus 1 includes conductors 2 that are adapted to rest on the patient's head 3. Therapy begins after a patient 4 presents in clinic and is taken to a clinical setting, which may include a quiet room in which lighting can be reduced, and in which the room contains a comfortable chair for the patient to use during therapy and which has adequate support for the patient's head 3 and neck. The conductors 2 are used to establish electrical contact between the apparatus 1 and the patient's head 3. Such contact may be enhanced by using cutaneous electrodes 5 and electrically conductive gel, both of which are well known in the art of electrophysiological measurements.

The conductors 2 are placed in a way such that the stimulation signal will pass through or proximate brain tissues to be stimulated. In various embodiments, such brain tissues may include, but are not limited to, the parietal lobes, somatosensory cortex, thalamus, prefrontal cortex, primary motor cortex, secondary motor cortex, insula or default mode network.

Once electrical contact is established, the room's lighting is typically reduced and the patient 4 is encouraged to close their eyes and relax. At that point, a clinical professional causes the apparatus 1 to start the therapy application, which starts a timer internal to the apparatus 1. The therapy application, using burst and rest sequences as described herein, is then applied according to the timing of the apparatus 1.

With specific reference to the embodiments, a method is provided for treating fibromyalgia, which is a chronic full body pain condition featuring centralized pain, and in many cases, underlying conditions that also cause nociceptive pain. A method is also provided for treating other central pain related neurological conditions, which include but are not limited to hyperalgesia, central pain, central sensitivity, chronic pain, abnormal brain network connectivity, neuropathic pain, central pain arising from chronic osteoarthritis, central pain arising from chronic back pain, chronic headache, migraine headache or depression. All of these conditions either feature or are believed to be causally related to centralized pain, which is further believed to be related to central sensitivity and abnormal neural network connectivity in pain processing areas of the brain.

The method includes the use of an electrical stimulation signal configured to reduce tissue impedance and deliver stimulation to brain tissues without being perceived by a patient 4 who is receiving the stimulation. In a preferred embodiment, the electrical stimulation signal comprises a tissue transmission signal further comprising a pulse train of frequency sufficient to reduce tissue impedance. Further, the individual pulses in the pulse train are pulse width modulated to create a variable duty cycle of on time and off time. Such pulse width modulation is conceived to control the time-averaged power delivered over an individual period of the pulses in the pulse train. Further still, the electrical stimulation signal comprises a therapeutic signal created by amplitude modulating the pulse train of the tissue transmission signal to create a waveshape comprising an amplitude envelope. In some embodiments, the waveshape forms a stimulation signal of at least one lower frequency compared with the frequency of the pulse train, and it is this lower frequency signal that acts as the therapeutic signal.

In one embodiment, the pulse train amplitudes and frequencies are chosen to be subthreshold for detection by a person being stimulated. In some instances of prior art, electrical stimulation methods are taught that teach avoidance of amplitudes and frequencies that are within the perceptible physiological stimulation range, that is, approximately between 1-300 Hertz (Hz), so as to prevent noxious sensations in a person receiving stimulation. However, the present invention teaches the combination of high frequency and low amplitude signals that are capable of providing stimulation within said physiological stimulation range that is not detected by a person receiving stimulation and still causes a therapeutic effect.

Such combinations of signal amplitudes and frequencies, in the embodiment, may include a pulse train with amplitude at a minimum value of 0 volts and a maximum value of 1 volt. In the preferred embodiment, the pulse train amplitude has a maximum value of 0.2 volts. Such combinations may also include a pulse train with frequencies between 10,000 Hz and 20,000 Hz. In the preferred embodiment, the pulse train frequency is approximately 15,000 Hz. This range of frequencies has been shown in tissue modeling studies to effectively overcome the impedances of the tissues of the head 3, thus allowing a stimulation signal applied noninvasively to pass through outer tissues such as the scalp and skull with less attenuation.

Further to the embodiment, the pulse train may be of any form, such as a monopolar waveform. Further still, the pulse train duty cycle may be between 20% and 60%. These duty cycles are anticipated to provide time-averaged power levels that are effective. In one exemplary embodiment, the pulse train duty cycle is approximately 37.5%.

Further still to the embodiments, the amplitude envelope formed by amplitude modulating the pulse train may form any waveshape. In one embodiment, the amplitude envelope may form a rectangular wave. Such rectangular waveshape may be advantageous as it has shown efficacy in clinical studies for treating pain in fibromyalgia. In another embodiment, the amplitude envelope forms a sinusoidal wave. In yet another embodiment, the amplitude envelope forms a composite of multiple sinusoidal waves. Such composite of multiple sinusoidal waves may be advantageous in that multiple lower stimulating frequencies are represented by a single waveshape.

In all embodiments, the waveshape is configured as a periodic waveform of frequencies sufficient to cause therapeutic electrical stimulation. In one embodiment, the waveshape has a frequency between 1 Hz and 30 Hz. This frequency range may also be used in embodiments involving multiple sinusoidal waves. This frequency range matches a range of frequencies commonly found in EEG signals in the human brain. In one embodiment, the waveshape has a frequency between 7 Hz and 12 Hz. This range provides stimulation in a range of frequencies commonly found in EEG signals in the human brain and representative of normal, alert conditions. In all embodiments, the frequency of the waveshape may be configured to change as a function of stimulation delivery time.

In one embodiment, a specific electrical stimulation method for treating fibromyalgia or other central pain related neurological conditions uses a pulse train which has a minimum amplitude value of 0 volts, a maximum value of 0.2 volts, a frequency of approximately 15,000 Hz and a duty cycle of approximately 37.5%. In the embodiment, the waveshape is a rectangular wave with frequencies ranging from 7 Hz to 12 Hz as a function of time. Further to the embodiment, the electrical stimulation signal is applied in sequences of bursts for at least two periods of time with at least one period of rest time comprising no stimulation in between each signal application. In one embodiment, EEG signal may be measured as part of the method. In one embodiment, EEG may be measured at the stimulation application site during the periods of rest. The measurement of EEG would be apparent to one ordinarily skilled in the art. Further to the embodiment, the bursts of stimulation signal may range in time from 1 second to 5 minutes. In another embodiment, the bursts of stimulation signal range in time from 30 seconds to 2 minutes. Further still to the embodiment, the periods of rest range in time from 1 second to 5 minutes. In another embodiment, the periods of rest are 60 seconds. Further still to the embodiment, EEG signal is measured for a period of time prior to a first burst of stimulation signal, with said period of time prior ranging from 1 second to 5 minutes. In a preferred embodiment, the period of time prior to a first burst is 3 minutes. Likewise, in still another embodiment, EEG signal is measured for a period of time after a final burst of stimulation signal, with said period of time prior ranging from 1 second to 5 minutes. In a preferred embodiment, the period of time after a final burst is 3 minutes.

In one embodiment, the electrical stimulation comprises a burst and rest time sequence consisting of a first three minute period of rest, a first burst of stimulation signal lasting 30 seconds, a second period of rest lasting 60 seconds, a second burst of stimulation signal lasting 60 seconds, a third period of rest lasting 60 seconds, a third burst of stimulation lasting 90 seconds, and a fourth period of rest lasting three minutes. In an exemplary embodiment, the stimulation signal burst is a pulse train that is amplitude modulated to form rectangular waveshapes, with said waveshapes having frequencies sweeping from 7 Hz to 12 Hz over approximately equal periods of time during the burst.

Further to all embodiments of the methods taught herein, the electrical stimulation signal is conducted along a current pathway extending through at least one portion of a brain. In one embodiment, such portions of a brain are selected from those portions that are involved in central pain. Such portions of a brain may include, but are not limited to, the parietal lobes, somatosensory cortex, thalamus, prefrontal cortex, primary motor cortex, secondary motor cortex, insula or default mode network. Further to the embodiment, the method is accomplished by using at least two electrical leads that are either invasive or noninvasive, and are placed proximate to the portion of a brain to be stimulated, such that a current pathway includes said portion of a brain. In one embodiment, such current pathway is provided by placing a first electrical lead proximate to the parietal lobes along the median plane of a human head 3, and a second electrical lead is placed proximate to the right ear of the head 3. In another embodiment, another current pathway is provided by placing a first electrical lead proximate to International 10-20 electrode placement site Pz on a head 3, and a second electrical lead is placed proximate to the right ear lobe of the head 3. The placement of electrical leads and accomplishment of electrical interface is readily known to those of ordinary skill in the art.

Further to the method, treating fibromyalgia or other central pain related neurological conditions consists of treatment applications of the electrical stimulation signal methods taught herein, wherein the treatment applications are applied repeatedly over a period of time to cause a lasting therapeutic effect. In one embodiment, treatment applications are applied at least once a day, with at least one day transpiring between treatment applications during which time no electrical stimulation is applied to the patient 4. In another embodiment, treatment applications are applied twice in a calendar week, with two days transpiring between treatment applications. Further to the embodiment, treatment is applied over a range of 8 to 24 weeks. In one exemplary embodiment, treatment comprises 24 treatment applications applied over a period of 12 consecutive weeks. The method is further accomplished when additional treatment applications are applied as necessary to further treat the fibromyalgia and achieve more satisfactory alleviation of symptoms.

In one embodiment, a method of treating fibromyalgia or other central pain related neurological conditions is accomplished using an electrical stimulation signal comprising a monopolar pulse train of frequency sufficient to reduce tissue impedance; wherein the pulse train amplitude has a minimum value of 0 volts, a maximum value of 0.2 volts, a frequency of approximately 15,000 Hz and a duty cycle of approximately 37.5%; wherein the pulse train is amplitude modulated to form a rectangular waveshape with frequencies ranging from 7 Hz to 12 Hz as a function of time; wherein each application of the electrical stimulation comprises the burst and rest time sequence consisting of a first three minute period of rest, a first burst of stimulation signal lasting 30 seconds with frequencies sweeping from 7 Hz to 12 Hz over approximately equal periods of time during the burst, a second period of rest lasting 60 seconds, a second burst of stimulation signal lasting 60 seconds with frequencies sweeping from 7 Hz to 12 Hz over approximately equal periods of time during the burst, a third period of rest lasting 60 seconds, a third burst of stimulation lasting 90 seconds with frequencies sweeping from 7 Hz to 12 Hz over approximately equal periods of time during the burst and a fourth period of rest lasting three minutes; wherein the electrical stimulation is conducted along a current pathway created between a first electrical lead placed proximate to International 10-20 site Pz, and a second electrical lead placed proximate to the right ear lobe; in which the treatment applications are applied twice in a calendar week, with two days transpiring between treatment applications; and in which the treatment applications are applied over a period of 12 consecutive weeks.

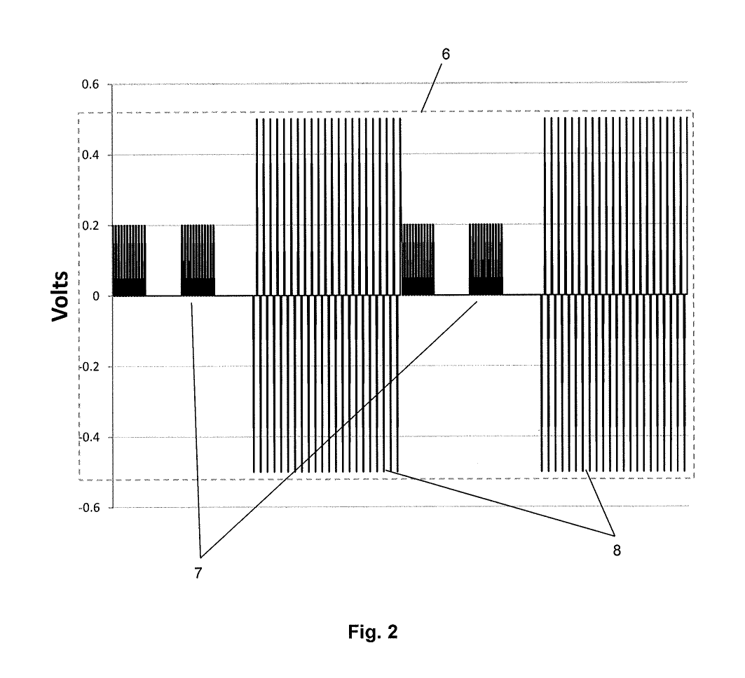

Further to the present invention, and with reference to FIG. 2, a method of treating a neurological condition is accomplished by using an electrical stimulation that is a composite signal 6 made up of multiple electrical signals. Such composite signal 6 may feature an electrical signal that has at least one signal form 7 configured to provide long-term treatment of the neurological condition by providing electrical stimulation that is therapeutic to physiological mechanisms involved in the neurological condition. An example of such physiological mechanisms may include abnormal brain conditions, such as excessive or insufficient neural network connectivity, and excessive or insufficient activity in brain regions associated with the neurological condition. Such composite 6 may further feature an electrical signal that has at least one signal form 8 configured to provide analgesia for short-term pain relief, whether the pain is associated with the neurological condition or not.

The advantage of such composite signals 6 is to provide multiple therapeutic benefits to a person receiving electrical stimulation. In particular, a person suffering from a neurological condition may experience multiple symptoms including pain. For example, a person suffering from fibromyalgia may experience pain, fatigue, stiffness, cognitive impairment, depression, anxiety, symptoms of irritable bowel syndrome and abnormal sensitivities. These symptoms may all be linked to central sensitivity, which may further be linked to an abnormal brain condition such as abnormal neural network connectivity, and may therefore be neurological in nature. A composite signal 6 as taught herein may include a signal form 7 configured to provide therapy to the brain mechanisms that are related to the neurological condition, and thus provide long-term benefit and symptom relief. The composite signal may also provide an additional signal form 8 configured to provide symptom relief that is relatively immediate in comparison. In the method, such additional signal form may be configured to provide short-term pain relief, regardless of whether the pain is nociceptive or central in nature. An example of such additional signal form may be one configured to modulate one or more neurotransmitters associated with pain or analgesia.

The practice of such composite electrical signals may be accomplished using any number of methods well known in the art, such as but not limited to the superposition of at least two individual signals. Such methods are known to a person ordinarily skilled in the art of electrical signal generation.

In one embodiment of the method, the at least one signal form 7 configured to provide long-term treatment of the neurological condition and the at least one signal form 8 configured to provide analgesia for short-term pain relief may be applied simultaneously during a treatment application. In another embodiment, the signal forms may be applied at alternating times during a treatment application.

Further to the embodiment, the at least one signal form 7 configured to provide long-term treatment of the neurological condition may comprise an electrical signal configured to treat an abnormal brain condition associated with the neurological condition, and the at least one signal form 8 configured to provide analgesia for short-term pain relief comprises an electrical signal configured to stimulate modulation of one or more neurotransmitters associated with analgesia. Such modulation may be selected from a group consisting of the release, expression, uptake, increased production, reduced production, inhibition or elimination of neurotransmitters. Such neurotransmitters may be selected from a group consisting of but not limited to serotonin, norepinephrine, glutamate, N-Methyl-D-aspartic acid, Substance P, gama-aminobutyric acid, various nucleotides or dopamine. Such neurological conditions may consist of but are not limited to fibromyalgia, central sensitivity, central pain, abnormal neural network connectivity, complex regional pain syndrome, phantom pain, irritable bowel syndrome, temporomandibular disorder, myofascial pain syndrome, regional soft-tissue pain syndrome, neuropathic pain, osteoarthritis, back pain, post-operative pain, depression, tension-type headaches or migraine headaches.

Further to the method, the at least one signal form 7 configured to provide long-term treatment of the neurological condition may comprise an electrical stimulation signal as taught herein configured to reduce tissue impedance and deliver stimulation to brain tissues without being perceived by a patient 4 who is receiving the stimulation. Further to the method, EEG signal may also be measured as part of the method and as taught herein. In one embodiment, EEG may be measured at the stimulation application site.

Further to the method, the at least one signal form 8 configured to provide analgesia for short-term pain relief may comprise a periodic signal such as, but not limited to, a pulse train or a sinusoidal waveform. In one embodiment, the periodic signal has frequencies between 1 Hz and 300 Hz, which is consistent with the physiologic stimulation frequency range as taught in some examples of the art. In another embodiment, the periodic signal has frequencies between 50 Hz and 150 Hz. Such frequencies have been shown to have the ability to modulate pain-related neurotransmitters. In exemplary embodiments, the periodic signal has a frequency of approximately 60 Hz or approximately 120 Hz. Such frequencies may relieve pain in fibromyalgia.

Further to the embodiments, the periodic signal may have amplitudes between −10 volts and +10 volts. In an exemplary embodiment, the periodic signal has minimum amplitude of −1 volts and maximum amplitude of +1 volt. Further still, the at least one signal form conceived to provide analgesia for short-term pain relief 8 comprises a direct current (DC) signal.

Further to the embodiments, the at least one signal form 8 configured to provide analgesia for short-term pain relief may be applied during periods of rest time before or after sequences of bursts of at least one signal form 7 configured to provide long-term treatment of a neurological condition.

Further to the method, the composite electrical stimulation signal 6 is conducted along at least one current pathway through at least one portion of a brain. Such portions of a brain may be selected from a group consisting of but not limited to the parietal lobes, somatosensory cortex, thalamus, prefrontal cortex, primary motor cortex, secondary motor cortex, insula or default mode network. Such current pathways may be created by placing at least two electrical leads proximate to the portion of a brain to be stimulated, as is previously taught herein. The method may also be accomplished by applying a composite electrical stimulation signal 6 using means that create at least a first current pathway through at least a first portion of a brain for providing long-term treatment of the neurological condition, and at least a second current pathway through at least a second portion of a brain for providing analgesia for short-term pain relief.

Further to the method, the treatment of a neurological condition may be accomplished by providing treatment applications of a composite electrical stimulation signal 6 applied repeatedly over a period of time to cause a lasting therapeutic effect. In one embodiment, treatment applications are applied at least once a day, with at least one day transpiring between treatment applications during which time no electrical stimulation is applied to the patient 4. In another embodiment, treatment applications are applied twice in a calendar week, with two days transpiring between treatment applications. Further to the embodiment, treatment is applied over a range of 8 to 24 weeks. In a preferred embodiment, treatment comprises 24 treatment applications applied over a period of 12 consecutive weeks. The method is further accomplished when additional treatment applications are applied as necessary to further treat the fibromyalgia and achieve more satisfactory alleviation of symptoms.

In a preferred embodiment, a method of treating a neurological condition is accomplished using a composite electrical stimulation signal 6 which comprises a signal 7 configured to provide long-term treatment of the neurological condition, and which further comprises a signal 8 configured to provide analgesia for short-term pain relief; wherein the signal 8 configured to provide analgesia for short-term pain relief comprises a train of both 60 Hz positive pulses and 60 Hz negative pulses, in which the positive pulses have a minimum amplitude of 0 volts and a maximum amplitude of 0.5 volts and the negative pulses have a maximum amplitude of 0 volts and a minimum amplitude of −0.5 volts, and in which the positive and negative pulses alternate and are equally spaced in time; wherein the signal 7 configured to provide long-term treatment of the neurological condition comprises a monopolar pulse train of frequency sufficient to reduce tissue impedance, in which the pulse train amplitude has a minimum value of 0 volts, a maximum value of 0.2 volts, a frequency of approximately 15,000 Hz and a duty cycle of approximately 37.5% and is amplitude modulated to form a rectangular waveshape with frequencies ranging from 7 Hz to 12 Hz as a function of time; wherein each application of the electrical stimulation comprises the burst sequence consisting of a first burst lasting three minutes and comprising the signal 8 configured to provide analgesia for short-term pain relief, a second burst lasting 30 seconds and comprising the signal 7 configured to provide long-term treatment of the neurological condition with frequencies sweeping from 7 Hz to 12 Hz over approximately equal periods of time during the burst, a third burst lasting 60 seconds and comprising the signal 8 configured to provide analgesia for short-term pain relief, a fourth burst lasting 60 seconds and comprising the signal 7 configured to provide long-term treatment of the neurological condition with frequencies sweeping from 7 Hz to 12 Hz over approximately equal periods of time during the burst, a fifth burst lasting 60 seconds and comprising the signal 8 configured to provide analgesia for short-term pain relief, a sixth burst lasting 90 seconds and comprising the signal 7 configured to provide long-term treatment of the neurological condition with frequencies sweeping from 7 Hz to 12 Hz over approximately equal periods of time during the burst, and a seventh burst lasting three minutes and comprising the signal 8 configured to provide analgesia for short-term pain relief; wherein the electrical stimulation is conducted along a current pathway created between a first electrical lead placed proximate to International 10-20 site Pz, and a second electrical lead placed proximate to the right ear lobe; in which the treatment applications are applied twice in a calendar week, with two days transpiring between treatment applications; and in which the treatment applications are applied over a period of 12 consecutive weeks.

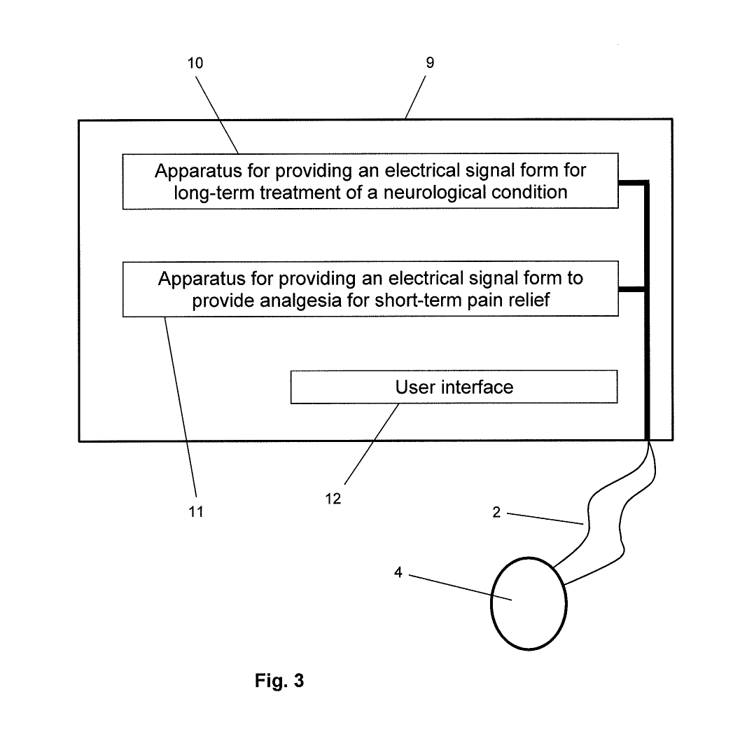

Further to the present invention, and with reference to FIG. 3, an electrical stimulation apparatus is provided comprising a composite electrical signal generator 9, with the composite electrical signal generator 9 further comprising at least one apparatus 10 adapted to generate an electrical signal form configured to provide long-term treatment of a neurological condition and further comprising at least one apparatus 11 adapted to generate an electrical signal form configured to provide analgesia for short-term pain relief.

In one embodiment, an apparatus 10 adapted to generate electrical stimulation signal forms configured to provide long-term treatment of a neurological condition is further configured to provide an electrical stimulation signal comprising a tissue transmission signal and a therapeutic signal. Further to the embodiment, an apparatus 11 adapted to generate electrical stimulation signal forms configured to provide analgesia for short-term pain relief further is further configured to provide an electrical stimulation signal configured to modulate one or more neurotransmitters associated with analgesia.

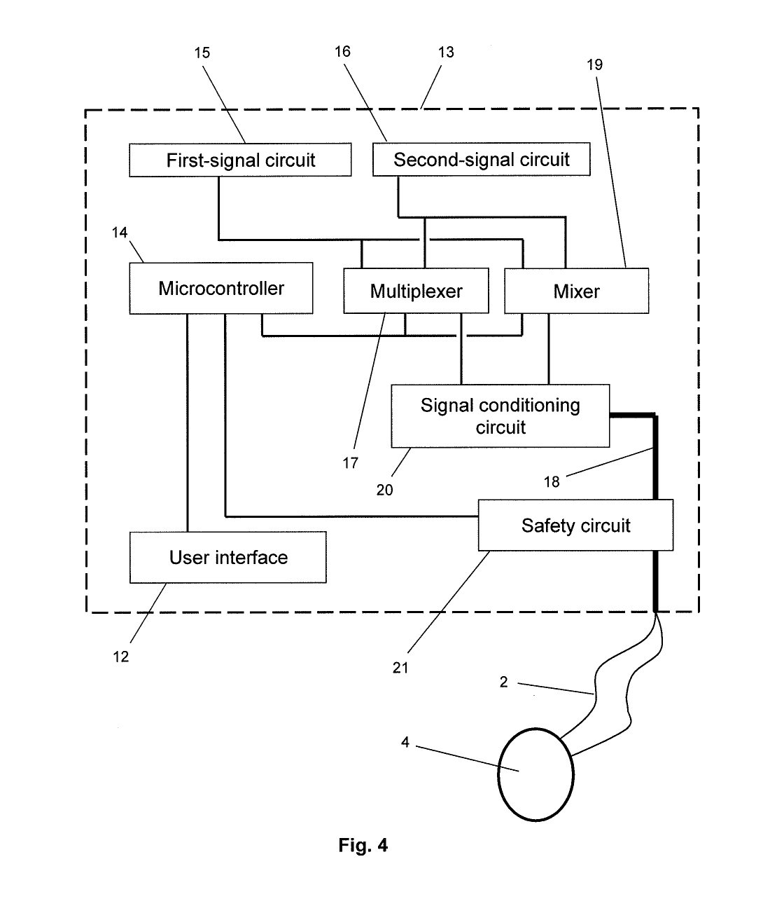

Numerous electrical circuit embodiments may be used to accomplish the apparatus. In one embodiment illustrated in FIG. 4, the composite electrical signal generator apparatus 13 may comprise a computing device such as a microcontroller 14 configured to manage timing of the therapy application and to control timed switching between periods of application of an electrical signal form configured to provide long-term treatment of a neurological condition, an electrical signal form configured to provide analgesia for short-term pain relief, and application of no signal to accomplish periods of rest.

The apparatus 13 may further comprise a first-signal circuit 15 configured to generate an electrical signal form configured to provide long-term treatment of a neurological condition. The apparatus 13 may further comprise a second-signal circuit 16 configured to generate an electrical signal form configured to provide analgesia for short-term pain relief.

The apparatus 13 may further comprise a multiplexer circuit 17, which may be controlled by the microcontroller 14 to selectively transmit output from either the first-signal circuit 15 or the second-signal circuit 16 onto a signal transmission conductor 18, or to transmit no signal to the signal transmission conductor 18 for accomplishing periods of rest. The apparatus 13 may further comprise a mixer circuit 19, which may be controlled by the microcontroller 14 to simultaneously transmit output from both the first-signal circuit 15 and the second-signal circuit 16 onto a signal transmission conductor 18, or to transmit no signal to the signal transmission conductor 18 for accomplishing periods of rest.

The embodiments might further comprise at least one signal conditioning circuit 20 configured to transform the generated signal waveforms into stimulation signals with desired voltage, amperage and power characteristics. Such signal conditioning circuit 20 may comprise an amplifier circuit. In the embodiments, the apparatus may further comprise any one or more circuit elements selected from but not limited to the group of circuit elements consisting of an EEG amplifier configured to measure EEG signals, a filter circuit configured to reduce electrical noise in EEG signals, an isolation amplifier configured to protect human subjects, an analog-to-digital interface configured to convert analog EEG signals to digital signals, and an isolated power supply configured to provide circuit power and human subject protection.

The embodiments may further comprise one or more conductors 2 electrically coupled to the signal transmission conductor 18 and configured to deliver the composite electrical signal from the composite electrical signal generator apparatus to a subject 4 being stimulated.

The embodiments may further comprise at least one safety circuit 21. In one such embodiment, a safety circuit 21 comprises a redundant timer circuit used to assure that an expected amount of stimulation time is not exceeded. Such safety circuit 21 may be used in case of a microcontroller 14 failure while the stimulation signal is being transmitted to the signal transmission conductor 18. Such safety circuit 21 might be configured to open the signal transmission conductor 18 if an expected maximum stimulation time is exceeded, thus preventing the subject 4 from receiving more stimulation than desired.

The embodiments may further comprise a user interface 12, such as but not limited to a software graphic user interface which provides user guidance for providing a composite stimulation signal.

The invention is not limited in any way to the embodiments disclosed herein. In this regard, no attempt is made to show structural details of the disclosed apparatuses or process details of the disclosed methods in more detail than is necessary for a fundamental understanding of the disclosed apparatuses and methods. The description is intended only to make apparent to those skilled in the art how the several forms of the invention may be embodied in practice.

### Example 1

A clinical trial entitled “The Clinical Effect of Low-Intensity Electromagnetic Field Neurostimulation in Fibromyalgia Syndrome Patients” (Hargrove et al. Pain Med. 2012 Jan; 13(1):115-24. ClinicalTrials.gov registration NCT01180244) was conducted at two independent sites. It utilized a randomized, double-blind sham controlled design with a subsequent unblinded crossover of sham treated patients.

The study was conducted on patients diagnosed with fibromyalgia using the American College of Rheumatology's 1990 (ACR 1990) classification criteria. Patients received either active treatment using a signal form taught herein, or a sham treatment. Treatment was applied twice a week for a period of eleven consecutive weeks. Total stimulation exposure at each treatment application was 3.0 minutes, for a total exposure time over the course of therapy of 66 minutes. A uniform protocol including signal form, exposure time and exposure location was applied during each study visit, as described below. Since the signal forms taught herein are not perceived by patients, the sham condition was created by simply not turning on the signal.

The specific protocol for the NCT01180244 study was as follows:

A total of 86 patients were enrolled in the study. Of these, no patients in the active treatment group voluntarily discontinued therapy. Seven patients in the sham group discontinued therapy citing no therapeutic benefit as the reason. One patient in the intervention group failed to return for end of therapy assessment and examination (i.e. was lost to follow-up), therefore no end of therapy data was gathered.

Eligibility of volunteers was assessed at screening that included a medical and surgical history, a physical examination, completion of patient self-report questionnaires designed to provide some of the study's outcome measures, an EEG test and a dolorimeter assessment of tender points (TePs) at 18 sites per the ACR 1990 classification criteria. Key subject inclusion criteria included having a confirmed diagnosis of primary fibromyalgia per the ACR 1990 classification criteria and ongoing symptoms lasting at least 48 months with no recent remission or significant change. Patients with other pain related diagnoses, chronic neurological disorder or significant systemic disorders were excluded from enrollment. Subjects with psychiatric disorders, other than depression and anxiety, requiring separate treatment were also excluded from enrollment.

Outcome measures included the Fibromyalgia Impact Questionnaire (FIQ), the number of positive tender points (TePs) (i.e. those tender points eliciting a painful response with less than 4 kg/cm2 dolorimeter pressure), and the tender point pressure pain threshold (PPT) derived by summing the dolorimetry pain thresholds for each of the 18 TePs assessed. In addition, all patients completed neuropsychiatric assessments, health outcomes questionnaires and a seven-question Sleep Visual Analog Scale (Sleep VAS) questionnaire.

Safety was evaluated by monitoring for patient-reported adverse events (AEs) during the study and at post-therapy examination. In addition, a subset of patients participating in long-term follow-up was asked to disclose any significant changes in health. No treatment emergent serious adverse effects were encountered over the course of the study. No adverse events or negative effects of treatment were discovered in long-term follow-up. Patient-reported AEs during the course of therapy were minor and resolved without medical intervention.

Efficacy was evaluated following repeat screening procedures on each patient conducted 1-2 weeks after the completion of therapy. These procedures included a physical examination, TeP and PPT evaluation, and repeat completion of outcome measures questionnaires. No assessment was done during the therapy period.

Table 1 provides a brief summary of outcomes data relevant to efficacy for the treatment of pain in fibromyalgia. Data provided is a measure of post-therapy to baseline differences presented as raw data, with percent change and p-values (statistical significance) in parentheses.

TABLE 1
Treatment outcomes related to pain in fibromyalgia

| | Intervention | Sham | |
|---|---|---|---|
| Outcome Measure | Group | Group | Conclusion |
| Post-therapy change and percent | −1.8 (27%, | −0.4 (7%, | Patient self-reported |
| improvement in pain VAS assessed | p < 0.001) | p = 0.20) | improvement in pain, as |
| by the FIQ, paired t-test individual | | | evidenced by changes in pain |
| averages (range 0-10) | | | VAS, shows significant |
| Post-therapy group difference in | 1.3 (20%, p = 0.02) | | improvement in the intervention |
| pain VAS assessed by the FIQ, t- | | | group, and no significant |
| test on group averages | | | change in the sham group. |
| | | | Group outcome differences are |
| | | | significant. |
| Post-therapy change and percent | −13.6 (22%, | −4.3 (7%, | Patient self-reported |
| improvement in overall FIQ score, | p < 0.001) | p = 0.054) | improvement in fibromyalgia- |
| paired t-test individual averages | | | specific symptoms, as |
| (range 0-100) | | | evidenced by changes in the |
| Post-therapy group difference in | 9.3 (15%, p = 0.01) | | overall FIQ score, shows |
| overall FIQ score, t-test on group | | | significant improvement in the |
| averages | | | intervention group, and no |
| | | | significant change in the sham |
| | | | group. Group outcome |
| | | | differences are significant. |
| Post therapy change and percent | −7.3 (42%, | −0.1 (1%, | The therapy resulted in |
| improvement in number of positive | p < 0.001) | p = 0.67) | significant improvement in the |
| TePs, paired t-test individual | | | number of tender points in the |
| averages (range 0-18) | | | intervention group, and no |
| Post-therapy group difference in | 6.9 (41%, p < 0.001) | | change in the sham group. |
| number of positive TePs, t-test on | | | Group outcome differences are |
| group averages | | | significant. |
| Post therapy change and percent | +19.1 (52%, | −2.7 (−7%, | The therapy resulted in |
| improvement in PPT, paired t-test | p < 0.001) | p = 0.04) | significant improvement in the |
| individual averages (range 9-72) | | | PPT in the intervention group. |
| Post-therapy group difference in | 21.8 (59%, p < 0.001) | | The sham group showed a |
| PPT, t-test on group averages | | | significant worsening in PPT (p = |
| | | | 0.04). Group outcome |
| | | | differences are significant. |
| Post-therapy change and percent | −105.8 | −30.0 (11%, | Patient self-reported |
| improvement in sleep VAS score, | (53%, | p = 0.13) | improvement in sleep, as |
| paired t-test individual averages | p < 0.001) | | evidenced by changes in the |
| (range 0-600) | | | sleep VAS score, shows |

| | | |
|---|---|---|
| Post-therapy group difference in | 75.8 (42%, p = 0.01) | significant improvement in the |
| sleep VAS score, t-test on group | | intervention group, and no |
| averages | | significant change in the sham |
| | | group. Group outcome |
| | | differences are significant. |

Table 2 reports group comparison statistics for 30 patients in the control (sham) group that underwent subsequent unblinded crossover to actual intervention. The electrical stimulation signal protocol used was the same as that used on the original active treatment group and taught herein. It should be noted that some of the patients in the control group chose not to participate in the unblinded crossover phase.

TABLE 2
Group comparison of unblinded crossover patients
(N = 30, t-test on group averages)

| | | | | | Post-Sham |
|---|---|---|---|---|---|
| | | | | Baseline to | to |
| | Baseline | Post-Sham | Crossover | Post-Sham | Crossover |
| Outcome Measure | Mean [SD] | Mean [SD] | Mean [SD] | p-value | p-value |
| FIQ Pain VAS score | 6.1 [2.4] | 5.9 [2.7] | 4.5 [2.8] | 0.84 | 0.06 |
| Overall FIQ score | 55.6 [17.3] | 50.9 [20.2] | 37.6 [20.9] | 0.38 | 0.03 |
| Number of positive TePs | 16.8 [1.9] | 16.6 [1.9] | 7.6 [4.6] | 0.79 | <0.001 |
| PPT, t-test on group | 38.9 [11.9] | 35.9 [9.5] | 61.0 [7.7] | 0.29 | <0.001 |
| averages | | | | | |

SD—standard deviation

Additional study data show that the electrical stimulation therapy also resulted in significant improvements in other symptoms associated with fibromyalgia. There were no site, evaluator or treating clinician effects that altered the results.

A follow-up study was completed with a cohort of study participants (Hargrove et al. Arch Phys Med Rehabil. 2012 October; 93(10):1868-71). The follow up included a symptom survey and the FIQ, and was intended to collect information concerning the long-term safety and efficacy of the electrical stimulation treatment taught herein.

Of the original 86 participants, 39 patients who were able to be contacted were mailed follow-up surveys. A series of Likert scale-based questions asked patients to report on symptom changes for tenderness, general pain, sleep, fatigue (energy), and depression/mood at both the end of study and currently. Patients were also asked to indicate how medicine use for both fibromyalgia pain and sleep had changed, and were asked a series of questions about their use of tricyclic antidepressants, selective serotonin reuptake inhibitors, pregabalin, duloxetine and milnacipran since completing the study. Patients were also asked to indicate how their need to visit physicians or other caregivers specifically for fibromyalgia changed.

Of 39 mailed surveys, 25 were returned (64%). The average respondent age was 59 years (range 39-71). All respondents were female. The average time since completion of therapy was 45 months (range 31-60).

In terms of efficacy, the mean total FIQ score for the respondent group was 52.6 at baseline, 35.7 at end of study and 31.8 at follow-up. One-way repeat measures ANOVA using the Holm-Sidak correction for multiple comparisons showed both end of study and follow-up scores were significantly different from baseline (P<0.001). Although follow-up scores were numerically superior to end of study scores, this difference was not significant (P=0.35). The FIQ score differences between baseline and end of study, and baseline and follow-up, were calculated for each respondent and found to be significantly correlated (R=0.78, P<0.001). Table 3 presents results of similar analyses performed on the FIQ's subscales.

TABLE 3
Analysis of FIQ outcomes

| | Respondent | | | Significance | | | |
|---|---|---|---|---|---|---|---|
| | FIQ Scores | | | (ANOVA) | | | |
| | | | 45-month | Baseline- | Baseline- | Correlation² | |
| | Baseline | EOS¹ | follow-up | EOS | follow-up | R-value | P-value |
| Total FIQ Score | 52.6 | 35.7 | 31.8 | P < 0.001 | P < 0.001 | 0.78 | <0.001 |
| Pain VAS | 6.0 | 4.0 | 3.6 | P < 0.01 | P < 0.001 | 0.78 | <0.001 |
| Fatigue VAS | 7.7 | 6.0 | 4.5³ | P < 0.01 | P < 0.001 | 0.69 | <0.001 |
| Sleep VAS | 7.9 | 6.2 | 5.0 | P < 0.01 | P < 0.001 | 0.51 | <0.01 |
| Stiffness VAS | 6.8 | 4.7 | 4.0 | P < 0.01 | P < 0.001 | 0.49 | 0.01 |
| Anxiety VAS | 4.0 | 1.8 | 2.6 | P < 0.001 | P = 0.03 | 0.70 | <0.001 |
| Depression VAS | 3.5 | 1.7 | 1.9 | P < 0.001 | P < 0.01 | 0.63 | <0.001 |

¹End of study
²Correlation between changes from baseline at EOS and at follow-up
³Long-term improvement since EOS is significant at P = 0.02

Table 4 provides a summary of patient responses for the level of improvement experienced in tenderness, pain, sleep, fatigue/energy, and depression/mood at end of study (EOS); their current condition for the same symptoms; and analysis of reported duration of response.

While there is an inherent bias in a survey method of this type, there does seem to be evidence of maintenance of improvement in fibromyalgia symptoms well beyond the end of the electrical stimulation treatment period. In addition to a suggestion of long-term symptom improvement, there was also evidence of reduction in both fibromyalgia medication usage and health care resource utilization. Of those who had reported using pain medicines, 76% reported either reduced or eliminated medicine use for pain. Of those that have taken sleep medicines, 44% reported either reduced or eliminated medicine use for sleep. Finally, 71% of all respondents indicate reduced or eliminated need to see physicians or caregivers for fibromyalgia treatment.

With regard to safety, patients were asked to report whether or not the therapy ever caused any pain or discomfort, and whether they experienced any side effects. No respondent reported experiencing any pain, discomfort or previously unreported side effects following completion of electrical stimulation therapy.

TABLE 4
Summary and analysis of patient responses for symptom improvement

| | | | | Fatigue | Depression/ |
|---|---|---|---|---|---|
| | Tenderness | Pain | Sleep | (energy) | mood |
| Respondent reported | | | | | |
| symptom change | | | | | |
| at EOS¹ | | | | | |
| Much improved | 50% | 36% | 25% | 44% | 32% |
| Somewhat improved | 38% | 44% | 42% | 32% | 20% |
| No change | 13% | 20% | 33% | 20% | 28% |
| Got worse | 0% | 0% | 0% | 4% | 4% |
| Does not apply | 0% | 0% | 8% | 0% | 16% |
| Reported state of | | | | | |
| symptoms at | | | | | |
| time of follow-up | | | | | |
| More improved | 20% | 28% | 13% | 36% | 32% |
| since EOS | | | | | |
| Same improvement | 36% | 36% | 46% | 20% | 28% |
| as EOS | | | | | |
| Improved for a time, | 40% | 24% | 8% | 24% | 12% |
| but returned | | | | | |
| Never changed/got | 4% | 12% | 29% | 20% | 12% |
| worse | | | | | |
| Does not apply | 0% | 0% | 4% | 0% | 16% |
| Duration of | | | | | |
| Symptom | | | | | |
| Improvements | | | | | |
| Reported^{2,3} | | | | | |
| Maintained to date | 62% | 75% | 87% | 74% | 80% |
| More than two years | — | 10% | 0% | 5% | — |
| 1-2 years | — | 5% | 7% | 5% | — |
| Less than one year | — | 10% | 0% | 5% | — |
| Unknown duration | 38% | 0% | 6% | 11% | 20% |

¹End of study
²Among long-term responders
³Incremental duration of effect was not evaluated for tenderness and depression/mood

Further, some of the survey's symptom questions gave respondents the option to report any worsening of symptoms. No respondent reported worsening of tenderness, pain or sleep (0%). A worsening of fatigue was reported by one patient (4%) and worsening of depression was reported by one other (4%).

Further to this study, EEG coherence analysis was performed to evaluate functional neural network connectivity in patient participants. Coherence, which is a measure of the correlation of relative amplitude and phase between pairs of EEG signals, is an established measure of functional network connectivity, and is ideally suited for providing temporally stable measures. Changes in EEG coherence in subjects who received the electrical stimulation treatment taught herein were compared to those subjects who received sham.

Procedurally, eyes-closed resting EEG was collected at 19 of the International 10-20 electrode sites for each subject at baseline and within one week of electrical stimulation therapy completion. To reduce coherence biasing due to cortical volume conduction over short spatial distances, only non-neighboring electrode pairings (N=118) were analyzed. Fibromyalgia symptomatology was assessed with the FIQ and the Short Form-36 (SF-36).

Baseline coherence was consistent between treatment groups in 112 of the 118 possible electrode pairs (95%, P<0.05). Analysis found that group mean change from baseline coherence decreased 67% at end of study in the active treatment group, while a corresponding decrease of only 18% was seen in the sham group (P<0.001). In the active treatment group, 79 out of 118 electrode pairings achieved at least a 50% mean change from baseline response, compared to only 9 in the sham group (χ2=86.3, P<0.001). Following active treatment using electrical stimulation methods as taught herein, a number of significant positive correlations in both inter- and intra-hemispherical electrode pairings were found between change from baseline coherence and improvements in total FIQ score and SF-36 domains. Further analysis showed improvements in FIQ score and pain VAS scale were greatest in subjects showing reductions in brain functional network connectivity based on changes in EEG coherence.

### Example 2

A second clinical trial entitled “A Double-Blinded, Randomized, Sham-Controlled, Proof of Concept Phase 2 Study Exploring the Safety and Efficacy of RINCE Technology for the Treatment of Patients with Fibromyalgia” (ClinicalTrials.gov registration NCT01825954), was conducted at a single site on 45 evaluable fibromyalgia patients. Like the trial of Example 1, this trial also utilized a randomized, blinded and sham controlled design. Fibromyalgia patient entry criteria were also similar to those used in Example 1.

The primary outcome measure for this study was the patients' pain level as assessed by 0-100 mm visual analog scale (VAS) recordings. At each visit, the patient recorded their 24-hour recall pain score and at every other visit they also provided their seven-day recall pain score. Additional outcome measures included the patient global impression of change (PGIC), the revised Fibromyalgia Impact Questionnaire (FIQ-R), the Jenkins sleep scale, the Beck depression inventory (BDI-II), the Multiple Ability Self-report Questionnaire (MASQ) and the Mental Clutter Scale (MCS).

The electrical stimulation signals applied to patients receiving active treatment included an electrical signal form configured for treating fibromyalgia or other central pain related neurological conditions as taught herein (i.e., “Signal 1”), a composite electrical stimulation signal configured to provide long-term treatment of a neurological condition and provide analgesia for short-term pain relief (i.e., “Signal 2”), and a signal configured only to provide analgesia for short-term pain relief (i.e., “Signal 3”).

Two separate groupings of therapeutic approach were studied. In a first grouping, 22 fibromyalgia patients were treated with either Signal 1 or a sham, essentially repeating the protocol of the trial of Example 1. Patients were randomized into three groups: Group 1A received 8 weeks of Signal 1 treatment followed by 4 weeks of sham; Group 1B received 12 weeks of Signal 1 treatment; and Group 1C received 12 weeks of sham. Sham consisted of no signal delivery. The daily treatment delivery schedule and signal delivery timing on all patients was the same as that used in the trial of Example 1.

In this first grouping of patients, primary outcomes analysis confirmed the therapeutic benefit which was also seen in the study of Example 1. Table 5 presents a summary of first grouping post-therapy to baseline differences in 24-hour pain VAS, comparing the various treatment groups. The table entries represent group mean raw changes in pain VAS measures.

TABLE 5
Analysis of 24-hour pain VAS outcomes in first patient grouping

| | Group 1B vs. | Group 1A vs. | Group 1B vs. |
|---|---|---|---|
| | Group 1C | Group 1C | Group 1A |
| Change in Pain VAS | −25.2 | −19.9 | −5.3 |
| P Value | 0.023 | 0.067 | 0.64 |

As seen in Table 5, the reduction in pain as evidenced by change in 24-hour pain VAS was significantly greater in patients receiving 12 weeks of Signal 1 treatment versus those patients receiving sham (P=0.023). Patients receiving 8 weeks of Signal 1 had a greater reduction in pain over those receiving sham, but the difference missed significance (P=0.067). The difference in pain reduction levels was not significant between patients receiving 12 weeks of Signal 1 versus those receiving 8 weeks of Signal 1 plus 4 weeks of sham (P=0.64), although the longer application of Signal 1 did produce numerically superior results.

In a second grouping, 23 additional fibromyalgia patients were treated with a combination of Signals 2 and 3. These patients were randomized into three groups: Group 2A received 8 weeks of the composite Signal 2 treatment followed by 4 weeks of the short-term analgesic Signal 3; Group 2B received 12 weeks of Signal 2 only; and Group 2C received 12 weeks of Signal 3 only. The daily treatment delivery schedule and signal delivery timing on all patients was the same as that used in the trial of Example 1 and in the first grouping of patients in Example 2.

Table 6 presents a summary of second patient grouping post-therapy to baseline differences in 24-hour pain VAS, comparing the various treatment groups. The table entries represent group mean raw changes in pain VAS measures.

TABLE 6
Analysis of 24-hour pain VAS outcomes in second patient grouping

| | Group 2B vs. | Group 2A vs. | Group 2B vs. |
|---|---|---|---|
| | Group 2C | Group 2C | Group 2A |
| Change in Pain VAS | +26.4 | +37.8 | −11.4 |
| P Value | −0.017 | −0.001 | 0.27 |

While all patients in this second grouping received pain reduction over baseline as a result of stimulation signal therapy, the data of Table 6 show that those patients receiving 12 weeks of only the short-term analgesic Signal 3 had greater pain reduction over those patients receiving 12 weeks of the composite Signal 2, with significance at P=0.017. Similarly, 12 weeks of short-term analgesic Signal 3 produced significantly greater pain reduction over those patients receiving 8 weeks of the composite Signal 2 followed by 4 weeks of the analgesic Signal 3 (P=0.001). Those patients receiving 12 weeks of only the composite signal had numerically greater pain reduction over those receiving 8 weeks of the composite signal followed by 4 weeks of the analgesic signal, however the difference was not significant (P=0.27).

The results from the second grouping of patients suggest that the use of a composite signal as taught herein can produce greater levels of immediate pain relief, as evidenced by 24-hour pain recall measures, over signals that are configured only for long-term treatment of fibromyalgia or other central pain related neurological conditions as taught herein. Further evidence of the mechanistic differences between the signals taught herein was gained by analyzing long-term patient outcomes. It would be expected that those patients who received only Signal 3, and who accordingly received the greatest level of pain reduction as evidenced by second grouping 24-hour pain VAS outcomes, would not have a corresponding long-term therapeutic effect. This would hypothetically be due to the lack of therapy with stimulation signals featuring components intended to provide long-term treatment of fibromyalgia or other central pain related neurological conditions, as taught herein. In support of this, follow up data was gathered from a cohort of patients studied in Example 2. In the cohort, second grouping patients who received only Signal 3 did not maintain any long-term pain reduction benefit. Their follow up pain VAS scores were on average 14.8% worse than they were at baseline. In comparison, the cohort of patients who received any level of Signals 1 and 2 were still on average 53.8% improved over baseline. The average time of follow up in these patients was approximately 4.5 months after cessation of therapy.

For both groupings, safety was assessed through routine collection of vital signs, routine collection of treatment emergent adverse events, and review of neurocognitive functioning results (MASQ and MCS) and mood scores (BDI-II). A consistent finding in the Example 2 study was an extremely low rate of adverse events related to the therapy. Consistent with the study of Example 1, there were very few adverse event categories that seemed to correlate with the stimulation therapy. Out of the patients enrolled in Example 2, there were no terminations for adverse events, and no serious adverse events. 97% of all adverse events were mild or moderate, and the vast majority appeared to be related to the underlying fibromyalgia condition. The only adverse event terms that may have been linked to therapy delivery were headache, vertigo and scalp dermatitis related to the use of electrically conductive gel at the interface between the patient's skin and the conductor's electrode.

What is claimed is:

1. A method of treating a neurological condition in a patient, the method comprising the step of applying an electrical stimulation from conductors to the patient's head at a stimulation application site, the electrical stimulation comprising a composite electrical signal further comprising at least one signal form configured to provide long-term treatment of the neurological condition and at least one signal form configured to provide analgesia for short-term pain relief.

2. The method of claim 1, in which the electrical stimulation is subthreshold for detection by the patient.

3. The method of claim 1, in which the at least one signal form configured to provide long-term treatment of the neurological condition and the at least one signal form configured to provide analgesia for short-term pain relief are applied simultaneously during a treatment application.

4. The method of claim 1, in which the at least one signal form configured to provide long-term treatment of the neurological condition and the at least one signal form configured to provide analgesia for short-term pain relief are applied at alternating times during a treatment application.

5. The method of claim 1, in which the at least one signal form configured to provide long-term treatment of the neurological condition comprises an electrical signal configured to treat an abnormal brain condition associated with the neurological condition.

6. The method of claim 1, in which the at least one signal form configured to provide analgesia for short-term pain relief comprises an electrical signal configured to stimulate modulation of one or more neurotransmitters associated with analgesia.

7. The method of claim 6, in which the modulation is selected from a group consisting of the release, expression, uptake, increased production, reduced production, inhibition or elimination of neurotransmitters.

8. The method of claim 6, in which the neurotransmitters are selected from a group consisting of serotonin, norepinephrine, glutamate, N-Methyl-D-aspartic acid, Substance P, gama-aminobutyric acid, various nucleotides or dopamine.

9. The method of claim 1, in which the condition is selected from a group of neurological conditions consisting of, but not limited to, fibromyalgia, central sensitivity, central pain, abnormal neural network connectivity, complex regional pain syndrome, phantom pain, irritable bowel syndrome, temporomandibular disorder, myofascial pain syndrome, regional soft-tissue pain syndrome, neuropathic pain, osteoarthritis, back pain, post-operative pain, depression, tension-type headaches or migraine headaches.

10. The method of claim 1, in which the at least one signal form configured to provide long-term treatment of the neurological condition comprises a tissue transmission component and a therapeutic component.

11. The method of claim 10, in which the tissue transmission component comprises a pulse train of frequency sufficient to reduce tissue impedance between the conductors and the patient's brain.

12. The method of claim 11, in which the pulse train amplitude has a minimum value of 0 volts and a maximum value of 1 volt.

13. The method of claim 11, in which the pulse train amplitude has a maximum value of 0.2 volts.

14. The method of claim 11, in which the pulse train frequency is between 10,000 Hz and 20,000 Hz.

15. The method of claim 11, in which the pulse train frequency is approximately 15,000 Hz.

16. The method of claim 11, in which the pulse train is monopolar.

17. The method of claim 11 in which the pulse train is pulse width modulated to create a variable duty cycle of on time and off time.

18. The method of claim 17, in which the pulse train duty cycle is between 20% and 60%.

19. The method of claim 17, in which the pulse train duty cycle is approximately 37.5%.

20. The method of claim 11, in which the pulse train is amplitude modulated to create a waveshape comprising an amplitude envelope.

21. The method of claim 20, in which the waveshape forms a therapeutic component.

22. The method of claim 20, in which the amplitude envelope forms a rectangular wave.

23. The method of claim 20, in which the amplitude envelope forms a sinusoidal wave.

24. The method of claim 20, in which the amplitude envelope forms a composite of multiple sinusoidal waves.

25. The method of claim 24, in which the multiple sinusoidal waves have individual frequencies between 1 Hz and 30 Hz.

26. The method of claim 20, in which the waveshape has a frequency between 1 Hz and 30 Hz.

27. The method of claim 20, in which the waveshape has a frequency between 7 Hz and 12 Hz.

28. The method of claim 20, in which the frequency of the waveshape changes as a function of stimulation delivery time.

29. The method of claim 20, in which the waveshape is a rectangular wave with frequencies ranging from 7 Hz to 12 Hz as a function of time.

30. The method of claim 11, in which the pulse train has an amplitude with minimum value of 0 volts and maximum value of 0.2 volts, a frequency of approximately 15,000 Hz and a duty cycle of approximately 37.5%.

31. The method of claim 1, in which the electrical stimulation includes a sequencing step in which an electrical signal is applied in bursts for at least two burst periods with at least one rest period comprising no stimulation in between each pair of bursts.

32. The method of claim 31, in which each burst period ranges in time from 1 second to 5 minutes.

33. The method of claim 31, in which each burst period ranges in time from 30 seconds to 2 minutes.

34. The method of claim 31, in which each rest period ranges in time from 1 second to 5 minutes.

35. The method of claim 31, in which each rest period is 60 seconds.

36. The method of claim 31, further comprising measuring the patient's EEG signal during the rest period.

37. The method of claim 36, in which the EEG is measured at the stimulation application site.

38. The method of claim 36, further comprising measuring the patient's EEG signal for a period of rest prior to a first burst of stimulation signal.

39. The method of claim 38, in which the period of rest prior to a first burst ranges in time from 1 second to 5 minutes.

40. The method of claim 38, in which the period of rest prior to a first burst is 3 minutes.

41. The method of claim 36, further comprising measuring the patient's EEG signal for a period of rest after a final burst of stimulation signal.

42. The method of claim 41, in which the period of rest after a final burst ranges in time from 1 second to 5 minutes.

43. The method of claim 41, in which the period of rest after a final burst is 3 minutes.

44. The method of claim 31, wherein the sequencing step comprises a burst and rest time sequence consisting of a first three minute period of rest;
applying a first burst of the electrical stimulation signal for 30 seconds;
after applying the first burst, ceasing application of the electrical stimulation signal for a second period of rest lasting 60 seconds;
after the second period of rest, applying a second burst of electrical stimulation signal for 60 seconds;
after applying the second burst, ceasing application of the electrical stimulation signal for a third period of rest lasting 60 seconds;
after the third period of rest, applying a third burst of the electrical stimulation signal for 90 seconds; and
after applying the third burst, ceasing application of the electrical stimulation signal for a fourth period of rest lasting three minutes.

45. The method of claim 44, further comprising measuring the patient's EEG signal during at least one period of rest.

46. The method of claim 44, in which the stimulation burst comprises a pulse train, wherein said pulses have amplitude having a minimum value of 0 volts, a maximum value of 0.2 volts, a frequency of approximately 15,000 Hz and a duty cycle of approximately 37.5%.

47. The method of claim 44, in which the stimulation signal burst is amplitude modulated to form rectangular waveshapes, with said waveshapes having frequencies sweeping from 7 Hz to 12 Hz over approximately equal periods of time during the burst.

48. The method of claim 31, further comprising performing the applying and sequencing steps repeatedly over a period of time.

49. The method of claim 48, in which the applying and sequencing steps are performed at least once a day, with at least one day transpiring between applications.

50. The method of claim 48, in which the applying and sequencing steps are performed twice in a calendar week, with two days transpiring between applications.

51. The method of claim 48, in which the applying and sequencing steps are performed repeatedly over a period ranging from 8 to 12 consecutive weeks.

52. The method of claim 48, in which the applying and sequencing steps are performed repeatedly over a period of 12 consecutive weeks.

53. The method of claim 48, in which the applying and sequencing steps are performed 24 times over the period of time.

54. The method of claim 53, further comprising performing the applying and sequencing steps additional times to further treat the neurological condition and achieve more satisfactory alleviation of symptoms.

55. The method of claim 1, in which the at least one signal form configured to provide analgesia for short-term pain relief comprises a periodic signal.

56. The method of claim 55, in which the periodic signal is a pulse train.

57. The method of claim 55, in which the periodic signal is a sinusoidal waveform.

58. The method of claim 55, in which the periodic signal has frequencies between 1 Hz and 300 Hz.

59. The method of claim 55, in which the periodic signal has frequencies between 50 Hz and 150 Hz.

60. The method of claim 55, in which the periodic signal has a frequency of approximately 60 Hz.

61. The method of claim 55, in which the periodic signal has a frequency of approximately 120 Hz.

62. The method of claim 55, in which the periodic signal has amplitudes between −10 volts and +10 volts.

63. The method of claim 55, in which the periodic signal has minimum amplitude of −1 volts and maximum amplitude of +1 volt.

64. The method of claim 1, in which the at least one signal form configured to provide analgesia for short-term pain relief is applied during periods of rest time before or after bursts of at least one signal form configured to provide long-term treatment of a neurological condition.

65. The method of claim 1, in which the at least one signal form configured to provide analgesia for short-term pain relief comprises a direct current (DC) signal.

66. The method of claim 1, in which the applying step includes the step of placing conductors to create a current pathway through at least one portion of the patient's brain.

67. The method of claim 66, in which the portion of a brain is selected from a group consisting of the parietal lobes, somatosensory cortex, thalamus, prefrontal cortex, primary motor cortex, secondary motor cortex, insula or default mode network.

68. The method of claim 66, wherein the conductors are placed proximate to the portion of the brain to be stimulated.

69. The method of claim 66, in which the conductors are noninvasive.

70. The method of claim 66, wherein the placing step comprises placing a first conductor proximate to the parietal lobes along the median plane, and a second conductor proximate to the right ear.

71. The method of claim 66, wherein the placing step comprises placing a first conductor proximate to International 10-20 site Pz, and a second conductor proximate to the right ear lobe.

72. The method of claim 1, wherein the applying step comprises applying electrical stimulation through conductors that create at least a first current pathway through at least a first portion of the patient's brain for providing long-term treatment of the neurological condition, and at least a second current pathway through at least a second portion of the patient's brain for providing analgesia for short-term pain relief.

73. The method of claim 72, wherein the conductors comprise at least two electrical leads placed proximate to the portion of the patient's brain to be stimulated.

74. The method of claim 73, in which the conductors are noninvasive.

75. A method of treating a neurological condition in a patient, the method comprising:
placing a first electrical lead on the patient's head proximate to International 10-20 site Pz;
placing a second electrical lead proximate to the patient's right ear lobe;
applying a composite electrical stimulation signal comprising a signal configured to provide long-term treatment of the neurological condition and a signal configured to provide analgesia for short-term pain relief;
wherein the signal configured to provide analgesia for short-term pain relief comprises a pulse train of both 60 Hz positive pulses and 60 Hz negative pulses;
wherein the positive pulses have a minimum amplitude of 0 volts and a maximum amplitude of 0.5 volts;
wherein the negative pulses have a maximum amplitude of 0 volts and a minimum amplitude of −0.5 volts;
wherein the positive and negative pulses alternate and are equally spaced in time;
wherein the signal to provide long-term treatment of the neurological condition comprises a monopolar pulse train of frequency sufficient to reduce tissue impedance;
wherein the pulse train amplitude has a minimum value of 0 volts, a maximum value of 0.2 volts, a frequency of approximately 15,000 Hz and a duty cycle of approximately 37.5%;
wherein the pulse train is amplitude modulated to form a rectangular waveshape with frequencies ranging from 7 Hz to 12 Hz as a function of time;
sequencing application of the electrical stimulation signal in a burst and rest time sequence consisting of;
a first burst comprising the signal configured to provide analgesia for short-term pain relief, lasting three minutes;
a second burst comprising the signal configured to provide long-term treatment of the neurological condition, lasting 30 seconds, with frequencies sweeping from 7 Hz to 12 Hz over approximately equal periods of time during the burst;
a third burst comprising the signal configured to provide analgesia for short-term pain relief, lasting 60 seconds;
a fourth burst comprising the signal configured to provide long-term treatment of the neurological condition, lasting 60 seconds, with frequencies sweeping from 7 Hz to 12 Hz over approximately equal periods of time during the burst;
a fifth burst comprising the signal configured to provide analgesia for short-term pain relief, lasting 60 seconds;
a sixth burst comprising the signal configured to provide long-term treatment of the neurological condition, lasting 90 seconds, with frequencies sweeping from 7 Hz to 12 Hz over approximately equal periods of time during the burst; and
a seventh burst comprising the signal configured to provide analgesia for short-term pain relief, lasting three minutes;
wherein the electrical stimulation is conducted along a current pathway created between the first electrical lead placed proximate to International 10-20 site Pz, and the second electrical lead placed proximate to the right ear lobe;
performing the placing, applying and sequencing steps twice in a calendar week, with at least one day transpiring between treatment applications, over a period of 12 consecutive weeks.

76. An electrical stimulation apparatus comprising an electrical signal generator adapted to provide an electrical signal form configured to provide long-term treatment of a neurological condition and to provide an electrical signal form configured to provide analgesia for short-term pain relief.

77. The apparatus of claim 76, in which the electrical signal generator further comprises at least one microcontroller configured to generate composite signal waveforms and coupled to at least one signal conditioning circuit configured to transform the composite signal waveforms into stimulation signals.

78. The apparatus of claim 77, in which the at least one signal conditioning circuit comprises an amplifier circuit.

79. The apparatus of claim 76, further comprising any one or more circuit elements selected from the group of circuit elements consisting of an EEG amplifier configured to measure EEG signals, a filter circuit configured to reduce electrical noise in EEG signals, an isolation amplifier configured to protect human subjects, an analog-to-digital interface configured to convert analog EEG signals to digital signals, and an isolated power supply configured to provide circuit power and human subject protection.

80. The apparatus of claim 76, further comprising at least two electrical leads for providing a composite stimulation signal current pathway between the electrical signal generator and a tissue to be stimulated.

81. The apparatus of claim 76, further comprising a user interface.

82. The apparatus of claim 81, further comprising a software graphic user interface which provides user guidance for providing a composite stimulation signal.

83. A method for treating a neurological condition in a patient, the method comprising: applying an electrical stimulation signal from conductors to the patient's head at a stimulation application site, wherein the electrical stimulation signal comprises a tissue transmission component and a therapeutic component; and sequencing the electrical stimulation signal in bursts for at least two burst periods with at least one rest period comprising no stimulation in between each pair of bursts.

84. The method of claim 83, in which the electrical stimulation signal is subthreshold for detection by the patient.

85. The method of claim 83, in which the tissue transmission component comprises a pulse train of frequency sufficient to reduce tissue impedance between the conductors and the patient's brain.

86. The method of claim 85, in which the pulse train amplitude has a minimum value of 0 volts and a maximum value of 1 volt.

87. The method of claim 85, in which the pulse train amplitude has a maximum value of 0.2 volts.

88. The method of claim 85, in which the pulse train frequency is between 10,000 Hz and 20,000 Hz.

89. The method of claim 85, in which the pulse train frequency is approximately 15,000 Hz.

90. The method of claim 85, in which the pulse train is monopolar.

91. The method of claim 85, in which the pulse train is pulse width modulated to create a variable duty cycle of on time and off time.

92. The method of claim 91, in which the pulse train duty cycle is between 20% and 60%.

93. The method of claim 91, in which the pulse train duty cycle is approximately 37.5%.

94. The method of claim 85, in which the pulse train is amplitude modulated to create a waveshape comprising an amplitude envelope.

95. The method of claim 94, in which the waveshape forms a therapeutic component.

96. The method of claim 94, in which the amplitude envelope forms a rectangular wave.

97. The method of claim 94, in which the amplitude envelope forms a sinusoidal wave.

98. The method of claim 94, in which the amplitude envelope forms a composite of multiple sinusoidal waves.

99. The method of claim 98, in which the multiple sinusoidal waves have individual frequencies between 1 Hz and 30 Hz.

100. The method of claim 94, in which the waveshape has a frequency between 1 Hz and 30 Hz.

101. The method of claim 94, in which the waveshape has a frequency between 7 Hz and 12 Hz.

102. The method of claim 94, in which the frequency of the waveshape changes as a function of stimulation delivery time.

103. The method of claim 94, in which the waveshape is a rectangular wave with frequencies ranging from 7 Hz to 12 Hz as a function of time.

104. The method of claim 85, in which the pulse train has an amplitude with minimum value of 0 volts and maximum value of 0.2 volts, a frequency of approximately 15,000 Hz and a duty cycle of approximately 37.5%.

105. The method of claim 83, in which each burst period ranges in time from 1 second to 5 minutes.

106. The method of claim 83, in which each burst period ranges in time from 30 seconds to 2 minutes.

107. The method of claim 83, in which each rest period ranges in time from 1 second to 5 minutes.

108. The method of claim 83, in which each rest period is 60 seconds.

109. The method of claim 83, further comprising measuring the patient's electroencephalogram (EEG) signal during the rest period.

110. The method of claim 109, in which the EEG is measured at the stimulation application site.

111. The method of claim 109, further comprising measuring the patient's EEG signal for a period of rest prior to a first burst of stimulation signal.

112. The method of claim 111, in which the period of rest prior to a first burst ranges in time from 1 second to 5 minutes.

113. The method of claim 111, in which the period of rest prior to a first burst is 3 minutes.

114. The method of claim 109, further comprising measuring the patient's EEG signal for a period of rest after a final burst of stimulation signal.

115. The method of claim 114, in which the period of rest after a final burst ranges in time from 1 second to 5 minutes.

116. The method of claim 114, in which the period of rest after a final burst is 3 minutes.

117. The method of claim 83, wherein the sequencing step comprises a burst and rest time sequence consisting of;
a first three minute period of rest;
applying a first burst of the electrical stimulation signal for 30 seconds;
after applying the first burst, ceasing application of the electrical stimulation signal for a second period of rest lasting 60 seconds;
after the second period of rest, applying a second burst of the electrical stimulation signal for 60 seconds;
after applying the second burst, ceasing application of the electrical stimulation signal for a third period of rest lasting 60 seconds;
after the third period of rest, applying a third burst of the electrical stimulation signal for 90 seconds; and
after applying the third burst, ceasing application of the electrical stimulation signal for a fourth period of rest lasting three minutes.

118. The method of claim 117, in which the stimulation burst comprises a pulse train, wherein said pulses have amplitude having a minimum value of 0 volts, a maximum value of 0.2 volts, a frequency of approximately 15,000 Hz and a duty cycle of approximately 37.5%.

119. The method of claim 117, in which the stimulation signal burst is amplitude modulated to form rectangular waveshapes, with said waveshapes having frequencies sweeping from 7 Hz to 12 Hz over approximately equal periods of time during the burst.

120. The method of claim 83, in which the applying step includes the step of placing conductors to create a current pathway through at least one portion of the patient's brain.

121. The method of claim 120, in which the portion of a brain is selected from a group consisting of the parietal lobes, somatosensory cortex, thalamus, prefrontal cortex, primary motor cortex, secondary motor cortex, insula or default mode network.

122. The method of claim 120, wherein the conductors are placed proximate to the portion of the brain to be stimulated.

123. The method of claim 120, wherein the placing step comprises placing a first conductor proximate to the parietal lobes along the median plane, and a second conductor proximate to the right ear.

124. The method of claim 120, wherein the placing step comprises placing a first conductor proximate to International 10-20 site Pz, and a second conductor proximate to the right ear lobe.

125. The method of claim 83, in which the conductors are noninvasive.

126. The method of claim 83, further comprising performing the applying and sequencing steps repeatedly over a period of time.

127. The method of claim 126, in which the applying and sequencing steps are performed at least once a day, with at least one day transpiring between applications.

128. The method of claim 126, in which the applying and sequencing steps are performed twice in a calendar week, with two days transpiring between applications.

129. The method of claim 126, in which the applying and sequencing steps are performed repeatedly over a period ranging from 8 to 24 consecutive weeks.

130. The method of claim 126, in which the applying and sequencing steps are performed repeatedly over a period of 12 consecutive weeks.

131. The method of claim 126, in which the applying and sequencing steps are performed 24 times over the period of time.

132. The method of claim 131, further comprising performing the applying and sequencing steps additional times to further treat the neurological condition and achieve more satisfactory alleviation of symptoms.

133. The method of claim 83, in which the neurological condition is selected from a group consisting of hyperalgesia, central pain, central sensitivity, chronic pain, abnormal brain network connectivity, neuropathic pain, central pain arising from chronic osteoarthritis, central pain arising from chronic back pain, chronic headache, migraine headache or depression.

134. A method of treating a neurological condition in a patient, the method comprising:
placing a first conductor on the patient's head proximate to International 10-20 site Pz;
placing a second conductor proximate to the patient's right ear lobe;
applying an electrical stimulation signal between the first and second conductors, the electrical stimulation signal comprising a tissue transmission component further comprising a monopolar pulse train of frequency sufficient to reduce tissue impedance between the conductors and the patient's brain;
wherein the pulse train amplitude has a minimum value of 0 volts, a maximum value of 0.2 volts, a frequency of approximately 15,000 Hz and a duty cycle of approximately 37.5%;
wherein the pulse train is amplitude modulated to form a rectangular waveshape with frequencies ranging from 7 Hz to 12 Hz as a function of time;
sequencing application of the electrical stimulation signal in a burst and rest time sequence consisting of:
a first three minute period of rest;
after the first period of rest, applying a first burst of the electrical stimulation signal for 30 seconds, with frequencies sweeping from 7 Hz to 12 Hz over approximately equal periods of time during the burst;
after applying the first burst, ceasing application of the electrical stimulation signal for a second period of rest lasting 60 seconds;
after the second period of rest, applying a second burst of the electrical stimulation signal for 60 seconds, with frequencies sweeping from 7 Hz to 12 Hz over approximately equal periods of time during the burst;
after applying the second burst, ceasing application of the electrical stimulation signal for a third period of rest lasting 60 seconds;
after the third period of rest, applying a third burst of the electrical stimulation signal for 90 seconds, with frequencies sweeping from 7 Hz to 12 Hz over approximately equal periods of time during the burst; and
after applying the third burst, ceasing application of the electrical stimulation signal for a fourth period of rest lasting three minutes;
performing the placing, applying and sequencing steps twice in a calendar week, with at least one day transpiring between treatment applications, over a period of 12 consecutive weeks.

135. A method for treating fibromyalgia in a patient, the method comprising: applying an electrical stimulation signal from conductors to the patient's head at a stimulation application site, wherein the electrical stimulation signal comprises a tissue transmission component and a therapeutic component; and sequencing the electrical stimulation signal in bursts for at least two burst periods with at least one rest period comprising no stimulation in between each pair of bursts.

136. The method of claim 135, in which the electrical stimulation signal is subthreshold for detection by the patient.

137. The method of claim 135, in which the tissue transmission component comprises a pulse train of frequency sufficient to reduce tissue impedance between the conductors and the patient's brain.

138. The method of claim 137, in which the pulse train amplitude has a minimum value of 0 volts and a maximum value of 1 volt.

139. The method of claim 137, in which the pulse train amplitude has a maximum value of 0.2 volts.

140. The method of claim 137, in which the pulse train frequency is between 10,000 Hz and 20,000 Hz.

141. The method of claim 137, in which the pulse train frequency is approximately 15,000 Hz.

142. The method of claim 137, in which the pulse train is monopolar.

143. The method of claim 137, in which the pulse train is pulse width modulated to create a variable duty cycle of on time and off time.

144. The method of claim 143, in which the pulse train duty cycle is between 20% and 60%.

145. The method of claim 143, in which the pulse train duty cycle is approximately 37.5%.

146. The method of claim 143, in which the pulse train is amplitude modulated to create a waveshape comprising an amplitude envelope.

147. The method of claim 146, in which the waveshape forms a therapeutic component.

148. The method of claim 146, in which the amplitude envelope forms a rectangular wave.

149. The method of claim 146, in which the amplitude envelope forms a sinusoidal wave.

150. The method of claim 146, in which the amplitude envelope forms a composite of multiple sinusoidal waves.

151. The method of claim 150, in which the multiple sinusoidal waves have individual frequencies between 1 Hz and 30 Hz.

152. The method of claim 146, in which the waveshape has a frequency between 1 Hz and 30 Hz.

153. The method of claim 146, in which the waveshape has a frequency between 7 Hz and 12 Hz.

154. The method of claim 146, in which the frequency of the waveshape changes as a function of stimulation delivery time.

155. The method of claim 146, in which the waveshape is a rectangular wave with frequencies ranging from 7 Hz to 12 Hz as a function of time.

156. The method of claim 137, in which the pulse train has an amplitude with minimum value of 0 volts and maximum value of 0.2 volts, a frequency of approximately 15,000 Hz and a duty cycle of approximately 37.5%.

157. The method of claim 135, in which each burst period ranges in time from 1 second to 5 minutes.

158. The method of claim 135, in which each burst period ranges in time from 30 seconds to 2 minutes.

159. The method of claim 135, in which each rest period ranges in time from 1 second to 5 minutes.

160. The method of claim 135, in which each rest period is 60 seconds.

161. The method of claim 135, further comprising measuring the patient's electroencephalogram (EEG) signal during the rest period.

162. The method of claim 161, in which the EEG is measured at the stimulation application site.

163. The method of claim 161, further comprising measuring the patient's EEG signal for a period of rest prior to a first burst of stimulation signal.

164. The method of claim 163, in which the period of rest prior to a first burst ranges in time from 1 second to 5 minutes.

165. The method of claim 163, in which the period of rest prior to a first burst is 3 minutes.

166. The method of claim 161, further comprising measuring the patient's EEG signal for a period of rest after a final burst of stimulation signal.

167. The method of claim 166, in which the period of rest after a final burst ranges in time from 1 second to 5 minutes.

168. The method of claim 166, in which the period of rest after a final burst is 3 minutes.

169. The method of claim 135, wherein the sequencing step comprises a burst and rest time sequence consisting of;
a first three minute period of rest;
applying a first burst of the electrical stimulation signal for 30 seconds;
after applying the first burst, ceasing application of the electrical stimulation signal for a second period of rest lasting 60 seconds;
after the second period of rest, applying a second burst of the electrical stimulation signal for 60 seconds;
after applying the second burst, ceasing application of the electrical stimulation signal for a third period of rest lasting 60 seconds;
after the third period of rest, applying a third burst of the electrical stimulation signal for 90 seconds; and
after applying the third burst, ceasing application of the electrical stimulation signal for a fourth period of rest lasting three minutes.

170. The method of claim 169, in which the stimulation burst comprises a pulse train, wherein said pulses have amplitude having a minimum value of 0 volts, a maximum value of 0.2 volts, a frequency of approximately 15,000 Hz and a duty cycle of approximately 37.5%.

171. The method of claim 169, in which the stimulation signal burst is amplitude modulated to form rectangular waveshapes, with said waveshapes having frequencies sweeping from 7 Hz to 12 Hz over approximately equal periods of time during the burst.

172. The method of claim 135, in which the applying step includes the step of placing conductors to create a current pathway through at least one portion of the patient's brain.

173. The method of claim 172, in which the portion of a brain is selected from a group consisting of the parietal lobes, somatosensory cortex, thalamus, prefrontal cortex, primary motor cortex, secondary motor cortex, insula or default mode network.

174. The method of claim 172, wherein the conductors are placed proximate to the portion of the brain to be stimulated.

175. The method of claim 172, wherein the placing step comprises placing a first conductor proximate to the parietal lobes along the median plane, and a second conductor proximate to the right ear.

176. The method of claim 172, wherein the placing step comprises placing a first conductor proximate to International 10-20 site Pz, and a second conductor proximate to the right ear lobe.

177. The method of claim 135, in which the conductors are noninvasive.

178. The method of claim 135, further comprising performing the applying and sequencing steps repeatedly over a period of time.

179. The method of claim 178, in which the applying and sequencing steps are performed at least once a day, with at least one day transpiring between applications.

180. The method of claim 178, in which the applying and sequencing steps are performed twice in a calendar week, with two days transpiring between applications.

181. The method of claim 178, in which the applying and sequencing steps are performed repeatedly over a period ranging from 8 to 24 consecutive weeks.

182. The method of claim 178, in which the applying and sequencing steps are performed repeatedly over a period of 12 consecutive weeks.

183. The method of claim 178, in which the applying and sequencing steps are performed 24 times over the period of time.

184. The method of claim 183, further comprising performing the applying and sequencing steps additional times to further treat the fibromyalgia and achieve more satisfactory alleviation of symptoms.

185. A method of treating fibromyalgia in a patient, the method comprising:
placing a first conductor on the patient's head proximate to International 10-20 site Pz;
placing a second conductor proximate to the patient's right ear lobe;
applying an electrical stimulation signal between the first and second conductors, the electrical stimulation signal comprising a tissue transmission component further comprising a monopolar pulse train of frequency sufficient to reduce tissue impedance between the conductors and the patient's brain;
wherein the pulse train amplitude has a minimum value of 0 volts, a maximum value of 0.2 volts, a frequency of approximately 15,000 Hz and a duty cycle of approximately 37.5%;
wherein the pulse train is amplitude modulated to form a rectangular waveshape with frequencies ranging from 7 Hz to 12 Hz as a function of time;
sequencing application of the electrical stimulation signal in a burst and rest time sequence consisting of:
a first three minute period of rest;
after the first period of rest, applying a first burst of the electrical stimulation signal for 30 seconds, with frequencies sweeping from 7 Hz to 12 Hz over approximately equal periods of time during the burst;
after applying the first burst, ceasing application of the electrical stimulation signal for a second period of rest lasting 60 seconds;
after the second period of rest, applying a second burst of the electrical stimulation signal for 60 seconds, with frequencies sweeping from 7 Hz to 12 Hz over approximately equal periods of time during the burst;
after applying the second burst, ceasing application of the electrical stimulation signal for a third period of rest lasting 60 seconds;
after the third period of rest, applying a third burst of the electrical stimulation signal for 90 seconds, with frequencies sweeping from 7 Hz to 12 Hz over approximately equal periods of time during the burst; and
after applying the third burst, ceasing application of the electrical stimulation signal for a fourth period of rest lasting three minutes;
performing the placing, applying and sequencing steps twice in a calendar week, with at least one day transpiring between treatment applications, over a period of 12 consecutive weeks.
